(19) United States
(12) Patent Application Publication
(10) Pub. No.: US 2016/0193325 A1
(43) Pub. Date: Jul. 7, 2016

(54) INFECTIOUS HEPACIVIRUS PSEUDO-PARTICLES CONTAINING FUNCTIONAL e1, e2 ENVELOPE PROTEINS

(71) Applicants: INSTITUT NATIONAL DE LA SANTE ET DE LA RECHERCHE MEDICALE (INSERM), Paris (FR); ECOLE NORMALE SUPERIEURE DE LYON, Lyon Cedex 07 (FR)

(72) Inventors: Birke Bartosch, Chuzelles (FR); Francois-Loic Cosset, Lyon (FR)

(21) Appl. No.: 15/066,888

(22) Filed: Mar. 10, 2016

Related U.S. Application Data
(63) Continuation of application No. 10/527,422, filed on Mar. 27, 2006, now abandoned.

(30) Foreign Application Priority Data
Sep. 13, 2002 (EP) 02292254.6
Mar. 3, 2003 (EP) 03290505.1

(51) Int. Cl. A61K 39/29; C07K 14/005; G01N 33/566; C12N 7/00

(52) U.S. Cl. CPC A61K 39/29; C12N 7/00; C07K 14/005; G01N 33/566; A61K 2039/5258

(57) ABSTRACT

The invention relates to the generation and the use of hepacivirus pseudo-particles containing native functional E1, E2 envelope glycoproteins assembled onto retroviral core particles. These particles are highly infectious and constitute a valid model of hepacivirus virion.

The invention relates to the generation and the use of hepacivirus pseudo-particles containing functional E1, E2 envelope glycoproteins assembled onto retroviral core particles. These particles are highly infectious and constitute a valid model of hepacivirus virion.

World-wide several hundred millions of people are infected with hepatitis C virus (HCV) (Lavanchy et al., 1999). Progression to chronic disease occurs in the majority of HCV infected persons. Infection is associated with an increased risk for liver diseases and hepato-cellular carcinoma and has become the main indication for liver transplantation. HCV infection also increases the number of complications in HIV infected people (Dieterich, 2002). No vaccine is currently available to prevent new infections and the only treatment for chronic hepatitis C is interferon-α therapy, either alone or in combination with the guanosine analogue ribavirin. However, only ˜40% of patients respond to treatment. Clearly, novel therapeutic strategies are urgently required as the health costs for HCV infected people are predicted to spiral dramatically in the next few decades.

Based upon the structure of its genome and mechanisms of replication, HCV has been regarded as the prototype for a new class of viruses and was tentatively classified within the Hepacivirus genus, within the Flaviviridae virus family (Robertson et al., 1998).

Hepacivirus proteins structural and non-structural proteins are expressed from a single polyprotein precursor and individually released in their respective cell compartments upon cleavage by cellular and viral proteases (Lindenbach and Rice, 2001). By analogy with other members of the Flaviviridae, hepacivirus, and in particular HCV genomic organization, suggests a virus consisting of a nucleocapsid comprising a viral genome and core protein (C) coated by a lipid envelop containing the two envelope glycoproteins E1 and E2.

So far, the study of hepatitis C virus (HCV) has been hampered by the lack of an efficient and reliable culture system for amplifying the virus and by the lack of suitable animal model to study HCV replication in vivo (Lindenbach and Rice, 2001). Recently, a model for HCV replication, based on the self-replication of engineered minigenomes in cell culture, has been established (Blight et al., 2000; Lohmann et al., 1999). Although very useful to study HCV genomic replication, this system does not support production of HCV particles (Pietschmann et al., 2002).

Production of HCV virus-like particles (VLP) in insect cells has already been reported (WO 98/21338, Wellnitz et al., 2002; Baumert et al.; 1998, Owsianka et al., 2001). However these particles were not secreted and their extraction from intracellular compartments yielded VLP preparations that were not infectious.

Pseudotyped Vesicular Stomatitis Virus (VSV) viral particles have also been engineered with chimeric E1 and/or E2 glycoproteins whose transmembrane domains were modified to allow their transport to the cell surface (Buonocore et al., 2002; Matsuura et al., 2001). However, such modifications are likely to disturb conformation and functions of the E1E2 complexes (Matsuura et al., 2001) and although such pseudo-particles stand among candidate HCV vaccines (Rose et al., 2001), their use as a tool to investigate HCV assembly and cell entry remains controversial since there is now a consensus that these previous results are artefactual (Buonocore et al., 2002).

Similarly, the international patent application WO 02/074941 discloses that lentiviral-based pseudo-particles displaying modified E1 and E2 HCV glycoproteins, which harbour alteration of their transmembrane domain, can infect 293 human kidney cells and HepG2 hepato-carcinoma cells. This patent application further shows that pseudo-particles derived from human foamy virus displaying unmodified E1E2 glycoproteins can also infect 293 and HepG2 cells. However, these finding are artefactual because, as stated above, transmembrane modifications of E1E2 loaded on HCV pseudo-particles (Hsu et al., 2003) abolishes their functional properties, and because wild-type HCV is not infectious for 293 as well as HepG2 cells (Bartosch et al., 2003a and b).

Chimeric HCV-BVDV (Bovine Viral Diarrhea Virus) particles were shown to be infectious for the human hepatocyte line Huh-7 (WO 00/75352). However, infectivity could not be neutralised by an anti-serum against HCV which indicated that these particles did not constitute a valid model of HCV virion.

New approaches are therefore sorely needed to study HCV assembly and cell entry in order to design HCV cell entry inhibitors and to study the humoral immune response against HCV. Availability of infectious, amplifiable HCV particles would also provide useful material for the development of diagnostic application as well as therapeutical drugs.

The inventors have successfully generated infectious pseudo-particles that were assembled by displaying functional, in particular unmodified, HCV glycoproteins onto retroviral and lentiviral core particles. The presence of a green fluorescent protein marker gene packaged within these HCV pseudo-particles allowed reliable and fast determination of infectivity mediated by the HCV glycoproteins. Primary hepatocytes as well as hepato-carcinoma cells were found to be the major targets of infection in vitro. Albeit low residual infectivity may be observed with pseudo-particles harbouring either E1 or E2 glycoprotein, high infectivity of the pseudo-particles required both E1 and E2 HCV glycoproteins. Infectivity was further found to be neutralized by sera from HCV-infected patients and by some anti-E2 monoclonal antibodies. Altogether, these results indicate that the pseudo-particles described herein are the first pseudo-particle reported so far to mimic the early infection steps of wild-type HCV (Bartosch et al., 2003a; Castet, 2003).

The invention thus overcomes proposes infectious hepacivirus pseudo-particles, and in particular HCV pseudo-particles harboring E1 and E2 glycoproteins, that constitute a valid model of hepacivirus virions.

### DEFINITIONS

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”. A common type of vector is a “plasmid”, which generally is a self-contained molecule of double-stranded DNA, usually of bacterial origin, that can readily accept additional (foreign) DNA and which can readily be introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence that initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme.

The term “transfection” means the introduction of a foreign nucleic acid (DNA, cDNA or RNA) into a cell so that the host cell will express the introduced gene or sequence to produce a desired substance, typically a protein coded by the introduced gene or sequence. The introduced gene may include regulatory or control sequences, such as start, stop, promoter, signal, secretion, or other sequences used by a cell's genetic machinery. A host cell that receives and expresses introduced DNA or RNA has been “transformed”.

The term “host cell” means any cell of any organism that is selected, modified, transformed, grown, or used or manipulated in any way, for the production of a substance by the cell, for example the expression by the cell of a gene, a DNA sequence, a protein, a virion. In the context of the invention, the host cell is a mammalian cell. Suitable host cells include Huh-7 human hepatocellular carcinoma (Nakabayashi et al., 1982), Hep3B human hepatocellular carcinoma (ATCC HB-8064), HepG2 human hepatocellular carcinoma (HB-8065), HT-1080 human fibrosarcoma (CCL-121), 293T human embryo kidney cells (ATCC CRL-1573), TE671 human rhabdomyosarcoma (ATCC CRL-8805), Jurkat human T cell leukemia (TIB-152), CEM human lymphoblastic leukemia (CCL-119), COS-7 African green monkey fibroblasts kidney (CRL-1651), VERO African green monkey kidney (CCL-81), PG-4 feline astrocyte (CRL-2032), BHK-21 golden hamster kidney (CCL-10), CHO Chinese hamster ovary (ATCC CCL-61), and NIH3T3 mouse fibroblasts. In a specific embodiment said host cell is 293T.

As used herein, the term “permissive cell” is meant for a cell that is permissive for a hepacivirus infection.

“Hepacivirus” denotes hepatitis C virus, GB viruses, i.e. GB virus A, GB virus B, GB virus C, and GBV-A like agents, and hepatitis G virus. Preferably, said hepacivirus is hepatitis C virus (HCV). In the context of the invention, said hepacivirus may be of any specie, genotype and subtype, where appropriate, and variants thereof.

“Hepatitis C Virus” or “HCV” is a member of the Flaviviridae family. HCV is the type specie of the genus hepacivirus. HCV genome, like other hepaciviruses genome, encodes a single polyprotein NH₂—C-E1-E2-P7-NS2-NS3-NS4a-NS4b-NS5a-NS5b-COOH that is processed co and post-translationally into both structural (N-terminal nucleocapsid protein termed “Core” (C), and glycoproteins E1 and E2) and non-structural (NS) proteins. The amino-terminal part of the polyprotein is cleaved by host cell proteases and its products, core and envelope (E1 and E2) proteins, are believed to be the major constituents of HCV particles (virions).

Although most cleavages in the polyprotein precursor proceed to completion during or immediately after translation, processing between E2 and p7, a hydrophobic domain found at the carboxy terminus of E2, is incomplete and results in the production of fully processed E2 and uncleaved E2-p7. The p7 polypeptide of HCV is a small hydrophobic protein which has not been well characterized yet. Indeed, the structure of this 63-amino-acid-long polypeptide has not been determined, and its putative function(s) remains unknown.

In the context of the invention, HCV is of any genotype, e.g. 1, 2, 3, 4, 5, 6, and subtype, e.g. a, b, c, d, e, f, g, h, k, and variants thereof.

The polyprotein sequence of HCV strain H (GenBank accession number AF009606) is shown in SEQ ID N° 16. Amino acid positions of HCV E1, E2, p7 and core proteins are indicated below by reference to this sequence SEQ ID N° 16.

The term “variant” refers to the homologous polynucleotide sequences and corresponding amino acid sequences found in the different HCV strains owing to HCV hypervariability.

Preferably, HCV has either 1a or 1 b genotype (Dubuisson et al., 1994) that stand among HCV genotypes that are the most prevalent and the most resistant to interferon-α therapy (Zein, 2000). Also preferably HCV has either genotype 2a, 2b, 3a, 3b, 4a or 4b.

The term “hepacivirus-like particles”, “hepacivirus-like pseudo-particles”, or “hepacivirus pseudo-particles”, or “hepacivirus pseudotype particles” as used herein, refer to non naturally occurring viral particles that comprise an envelope protein of an hepacivirus. Preferably, the hepacivirus-like particles according to the invention display the same cellular tropism as the wild-type hepacivirus virion.

The term “HCV-like particles” or “HCV pseudo-particles” or “HCV pseudotype particles” as used herein refers to non naturally occurring viral particles that comprise an envelope protein of HCV.

The particles of the invention more particularly comprise retroviral core proteins. Such particles may be readily produced by one skilled in genetic engineering techniques. One can for instance refer to EP 1 201 750 that describes production of synthetic retroviral particles expressing an antigen for modulating an immune response.

The hepacivirus, in particular HCV, pseudo-particles of the invention are infectious for a target cell. Preferably the HCV pseudo-particles display the same cellular tropism as wild-type HCV virion (also called “tropism of wild-type HCV virion”), i.e. a preferential tropism for hepatic cells. Preferably, an hepatic target cell may be selected from the group consisting of a primary human hepatocyte, and a cell from an hepatocarcinoma cell line HuH7, PLC/PRF/5, or Hep3B, and an HepG2 cell genetically engineered to express CD81 (“HepG2-CD81”). As stated above, wild-type HCV is not infectious for the HepG2 cell line, and as described herein, the HepG2 cell line shows a very weak level of infection with the HCV pseudo-particles of the invention. However, the inventors demonstrated that expression of CD81 is sufficient to restore HCV pseudo-particles entry in HepG2 cells (Bartosch et al., 2003b). HepG2-CD81 thus represents a further example of hepatic target cell for HCV virions and HCV pseudo-particles.

In the context of the invention, the term “infectious” is used to describe the capacity of the particles of the invention to complete the initial steps of viral cycle that lead to cell entry. However, upon interaction with the host cell, hepacivirus-like particles may or may not produce progeny viruses.

The term “an envelope protein of a hepacivirus” denotes the native E1 or E2 glycoprotein of a hepacivirus, or a mutant thereof.

By an “E1 glycoprotein” or “E1 protein” is meant an envelope 1 protein (E1) from any specie, genotype, subtype and variants of hepacivirus strains.

By an “E2 glycoprotein” or “E2 protein” is meant an envelope 2 protein (E2) from any specie, genotype, subtype and variants of hepacivirus strains.

Preferably, E1 and E2 glycoproteins are derived from a same hepacivirus strain. Preferably, E1 and/or E2 glycoproteins are native.

By a “p7 protein” is meant a native p7 protein, or a mutant thereof, from any specie, genotype, subtype and variants of hepacivirus strains. Preferably, p7 protein and E1 and/or E2 glycoproteins are derived from a same hepacivirus strain. Preferably, p7 protein and E1 and/or E2 glycoproteins are native.

The term “an envelope protein of HCV” denotes the E1 or E2 glycoprotein of HCV, or a mutant thereof.

By a “HCV E1 glycoprotein” or “HCV E1 protein” is meant an envelope 1 protein (E1) from any genotype, subtype and variant of HCV strains. Full length E1 protein is defined by amino acids 192 to 383 of SEQ ID N° 16.

By a “HCV E2 glycoprotein” or “HCV E2 protein” is meant an envelope 2 protein (E2) from any genotype subtype and variant of HCV strains. Full length E2 protein is defined by amino acids 384 to 746 of SEQ ID N° 16.

Preferably, HCV E1 and E2 glycoproteins are derived from a same HCV strain. Preferably, HCV E1 and/or E2 glycoproteins are native.

By a “HCV p7 protein” is meant a native p7 protein (amino acids 747 to 809 as shown in SEQ ID N° 16), or a mutant thereof, from any genotype, subtype and variant of HCV strains. Preferably, HCV p7 protein and E1 and/or E2 glycoproteins are derived from a same HCV strain. Preferably, HCV p7 protein and E1 and/or E2 glycoproteins are native.

The term “mutant” or “mutation” is meant for alteration of the DNA sequence that result in a modification of the amino acid sequence of native E1, E2, or p7 proteins. Such a modification can be for instance the substitution and/or deletion of one or more amino acids. Mutants notably include fragments of native E1, E2 and p7 proteins. Variants are particular examples of naturally occurring mutants. Mutants are more particularly contemplated as useful for identifying the structural elements of E1 and/or E2 proteins, and optionally p7 protein, necessary for maintaining cell infectivity or for increasing E1 and/or E2 antigenicity for vaccination purposes.

Preferably, i) a mutant E1, E2, or p7 protein retains the capacity to assemble on the retroviral-based pseudo-particles and makes it possible for the pseudo-particles so produced to be released by the host cell; and/or ii) the pseudo-particles harbouring a mutant E1, E2, or p7 protein retains the preferential tropism of HCV virion for hepatic cells.

In this regards, substantial modifications of the C-terminal transmembrane domain of E1 or E2 glycoprotein, which allows anchoring of the glycoprotein to the membrane and thus assembly on the viral particle, should preferably be avoided as they may result in an alteration of cellular tropism and/or prevent pseudo-particle assembly and release. More specifically, mutated E1 or E2 protein wherein the transmembrane domain and/or cytoplasmic tail has been substituted, at least partially, preferably substantially totally, with the transmembrane domain/cytoplasmic tail of a heterologous protein, such as the VSV-G protein, should be excluded from the scope of the invention, in particular when the pseudo-particles so produced no longer display the tropism of wild-type HCV virion.

Advantageously, hepacivirus pseudo-particles with mutated E1, E2, or p7 protein are those that do not show decreased infectiosity compared with the corresponding pseudo-particle harbouring native E1, E2, or p7 protein. In this respect, preferred hepacivirus pseudo-particles do not contain mutations affecting a domain of E1 or E2 which is implicated in hepacivirus attachment to a cell receptor. In particular, said pseudo-particle may not comprise a HCV E2 glycoprotein mutated in one of the binding domains to CD81 or Scavenger Receptor class B type 1 (SR-B1), which are putative HCV cell receptors (Pileri et al., 1998; Scarselli et al., 2002). E2 binding domain to CD81 has been defined as three discrete segments at both ends of E2 (amino-acids 474-494, 522-551 and 612-620 of SEQ ID N° 16) that may join together in the head-to-tail model of E2 glycoprotein homodimer (Yagnik et al., 2000) whereas E2 bonding to SR-B1 is mediated by the hypervariable region 1 (HVR1, amino acids 384 to 410 of SEQ ID N° 16) (Bartosch et al., 2003b; Scarselli et al., 2002).

Preferred mutations of E1, E2 or p7 protein are those that result in increased infectivity of the hepacivirus pseudo-particles harbouring them. In particular, said E2 mutant may be a E2 glycoprotein in which the last C-terminal residue is deleted (Ala 746 as shown in SEQ ID N° 16). A 10 to 50 fold enhancement of infectiosity was observed with the HCV pseudo-particles harbouring this C-terminally truncated E2 protein (amino acids 384 to 745 of SEQ ID N° 16) compared with HCV pseudo-particles harbouring the native, full length E2 glycoprotein.

However, a hepacivirus pseudo-particle according to the invention may contain mutated E1, E2, or p7 protein and show decreased infectiosity compared with the corresponding pseudo-particle harbouring native E1, E2, or p7 protein. Such pseudo-particles are nonetheless useful for vaccination purposes and represent an interesting tool for the comprehension of mechanisms for virus entry in cells. Accordingly, the mutants may encompass a E2 glycoprotein wherein hypervariable region 1 (HVR1) has been deleted, while the particles so produced remain infectious, albeit with a decreased infectiosity.

The term “hepacivirus core” is meant for a native, full length, core protein of a hepacivirus strains, a fragment thereof, or a variant thereof. According to an embodiment, the core protein is a N-terminally truncated form of hepacivirus core (ΔC) that comprises the core signal peptide. Upon completion of its addressing function, the core protein is processed by a cellular protease and thereby cleaved from the polyprotein E1E2. Accordingly, the hepacivirus core protein is not found in the pseudo-particles according to the invention. The one skilled in the art will readily understand that the hepacivirus core protein can be replaced with a peptide sequence containing the signal sequence of any heterologous type I membrane protein (i.e. a protein anchored to the membrane by its C-terminus).

“Signal peptide” is intended for a peptide present on proteins that are destinated either to be secreted or to be membrane components. The signal peptide is usually located at the N-terminus and normally absent from the mature protein. It normally refers to the sequence (about 20 amino acids long) that interacts with signal recognition particle and directs the ribosome to the endoplasmic reticulum where co-translational insertion takes place. The signal sequence is normally removed from the growing peptide chain by signal peptidase, a specific protease located on the cisternal face of the endoplasmic reticulum.

In the context of the invention, “heterologous” is intended for a protein that is non-naturally occurring in the hepacivirus virion, and in particular in the HCV virion.

Examples of signal sequence from type I membrane protein are well known by the one skilled in the art. Reference may be made for instance to the signal sequence of immunoglobulins, or to signal sequences of viral type I glycoproteins, in particular retroviral surface glycoproteins. Examples of type I glycoproteins are further described in Paetzel et al. (2002) and von Heijne (1990).

As used herein, the term “HCV core” denotes a native core protein of the various HCV strains, a fragment thereof, or a variant thereof. The HCV core provides a signal peptide for the E1 or optionally E2 linked thereto that allows protein translocation to the endoplasmic reticulum. HCV core signal peptide corresponds to the last 21 residues of the carboxy-terminus of HCV core (GCSFSIFLLALLSCLTVPASA, SEQ ID N° 1; which corresponds to amino acids 171 to 191 of SEQ ID N° 16). According to an embodiment, the core protein is thus a N-terminally truncated form of HCV core (ΔC). Preferably ΔC comprises the last 21 residues of the carboxy-terminus of HCV core. In particular, HCV core may consist in the last 60 residues of the carboxy-terminus of HCV core (amino acids 132 to 191 of SEQ ID N° 16).

In the context of the invention, the terms “native” or “unmodified” are indifferently used to describe a wild-type, full-length protein.

The term “polyprotein” as used herein is used to describe a protein construct made up of individual proteins that are joined together in a sequence whereby they retain their original relevant biological activities.

The term “a polyprotein comprising a hepacivirus core protein linked to hepacivirus E1 protein and/or hepacivirus E2 protein”, or “a polyprotein comprising successively a hepacivirus core protein, and a hepacivirus E1 protein and/or hepacivirus E2 protein” includes the CE1E2, CE2E1, CE1, CE2, ΔCE1E2, ΔCE2E1, ΔCE1, and ΔCE2 polyproteins. Optionally, said polyproteins further contain the p7 protein. The polyprotein comprising a hepacivirus core protein linked to hepacivirus E1 protein and/or hepacivirus E2 protein thus additionally includes the CE1E2p7, CE2p7E1, CE1p7, CE2p7, ΔCE1E2p7, ΔCE2p7E1, ΔCE1p7, and ΔCE2p7 polyproteins.

“CE1E2” denotes a polyprotein comprising successively a hepacivirus core protein, a hepacivirus E1 protein and a hepacivirus E2 protein. “CE2E1” denotes a polyprotein comprising successively a hepacivirus core protein, a hepacivirus E2 protein and a hepacivirus E1 protein. “CE1” denotes a polyprotein comprising a hepacivirus core protein linked to a hepacivirus E1 protein. “CE2” denotes a polyprotein comprising a hepacivirus core protein linked to a hepacivirus E2 protein. “ΔCE1E2” denotes a polyprotein comprising a carboxy terminus of hepacivirus core protein, and hepacivirus E1 and hepacivirus E2 proteins. “ΔCE2E1” denotes a polyprotein comprising a carboxy terminus of hepacivirus core protein, and hepacivirus E2 and hepacivirus E1 proteins. “ΔCE1” denotes a polyprotein comprising a carboxy terminus of hepacivirus core protein, linked to hepacivirus E1 protein. “ΔCE2” denotes a polyprotein comprising a carboxy terminus of hepacivirus core protein, linked to hepacivirus E2 protein. ΔCE1E2, as well as ΔCE2, have been built by inserting a stop codon at the end of E2, whereas ΔCE2E1 and ΔCE1 have been built by inserting a stop codon at the end of E1. “CE1E2p7” denotes a polyprotein comprising successively a hepacivirus core protein, a hepacivirus E1 protein, a hepacivirus E2 protein, and a hepacivirus p7 protein. “CE2p7E1” denotes a polyprotein comprising successively a hepacivirus core protein, a hepacivirus E2 protein, a hepacivirus p7 protein, and a hepacivirus E2 protein. “CE1p7” denotes a polyprotein comprising successively a hepacivirus core protein, a hepacivirus E1 protein, and a hepacivirus p7 protein. “CE2p7” denotes a polyprotein comprising successively a hepacivirus core protein, a hepacivirus E2 protein, and a hepacivirus p7 protein. “ΔCE1E2p7” denotes a polyprotein comprising a carboxy terminus of hepacivirus core protein, a hepacivirus E1 protein, a hepacivirus E2 protein, and a hepacivirus p7 protein. “ΔCE2p7E1” denotes a polyprotein comprising a carboxy terminus of hepacivirus core protein, a hepacivirus E2 protein, a hepacivirus p7 protein and a hepacivirus E1 protein. “ΔCE1p7” denotes a polyprotein comprising a carboxy terminus of hepacivirus core protein, a hepacivirus E1 protein, and a p7 protein. “ΔCE2p7” denotes a polyprotein comprising a carboxy terminus of hepacivirus core protein, a hepacivirus E2 protein, and a p7 protein. ΔCE1E2p7, ΔCE1p7 and ΔCE2p7, have been built by inserting a stop codon at the end of p7 whereas ΔCE2p7E1 has been built by inserting a stop codon at the end of E1.

The term “a polyprotein comprising a HCV core protein linked to HCV E1 protein and/or HCV E2 protein”, or “a polyprotein comprising successively a HCV core protein and a HCV E1 protein and/or a HCV E2 protein”, includes the HCV CE1E2, CE2E1, CE1, CE2, ΔCE1E2, 10E2E1, ΔCE1, and ΔCE2 polyproteins. Optionally, said polyproteins further contain the p7 protein. The polyprotein comprising a HCV core protein linked to HCV E1 protein and/or HCV E2 protein thus additionally includes the HCV CE1E2p7, CE2p7E1, CE1p7, CE2p7, ΔCE1E2p7, ΔCE2p7E1, ΔCE1p7, and ΔCE2p7 polyproteins. “HCV CE1E2” denotes a polyprotein comprising successively a HCV core protein, a HCV E1 protein and a HCV E2 protein. “HCV CE2E1” denotes a polyprotein comprising successively a HCV core protein, a HCV E2 protein and a HCV E1 protein. “HCV CE1” denotes a polyprotein comprising a HCV core protein linked to a HCV E1 protein. “HCV CE2” denotes a polyprotein comprising a HCV core protein linked to a HCV E2 protein. “HCV ΔCE1E2” denotes a polyprotein comprising a carboxy terminus of HCV core protein, and HCV E1 and HCV E2 proteins. “HCV ΔCE2E1” denotes a polyprotein comprising a carboxy terminus of HCV core protein, and HCV E2 and HCV E1 proteins. “HCV ΔCE1” denotes a polyprotein comprising a carboxy terminus of HCV core protein, and a HCV E1 protein. “HCV ΔCE2” denotes a polyprotein comprising a carboxy terminus of HCV core protein, and a HCV E2 protein. HCV ΔCE1E2, as well as HCV ΔCE2, have been built by inserting a stop codon at the end of E2, whereas ΔCE2E1 and ΔCE1 have been built by inserting a stop codon at the end of E1. “HCV CE1E2p7” denotes a polyprotein comprising successively a HCV core protein, a HCV E1 protein, a HCV E2 protein, and a HCV p7 protein. “HCV CE2p7E1” denotes a polyprotein comprising successively a HCV core protein, a HCV E2 protein, a HCV p7 protein, and a HCV E2 protein. “HCV CE1p7” denotes a polyprotein comprising successively a HCV core protein, a HCV E1 protein, and a HCV p7 protein. “HCV CE2p7” denotes a polyprotein comprising successively a HCV core protein, a HCV E2 protein, and a HCV p7 protein. “HCV ΔCE1E2p7” denotes a polyprotein comprising a carboxy terminus of HCV core protein, a HCV E1 protein, a HCV E2 protein, and a HCV p7 protein. “HCV ΔCE2p7E1” denotes a polyprotein comprising a carboxy terminus of HCV core protein, a HCV E2 protein, a HCV p7 protein, and a HCV E1 protein. “HCV ΔCE1p7” denotes a polyprotein comprising a carboxy terminus of HCV core protein, a HCV E1 protein, and a p7 protein. “HCV ΔCE2p7” denotes a polyprotein comprising a carboxy terminus of HCV core protein, a HCV E2 protein, and a p7 protein. HCV ΔCE1E2p7, HCV ΔCE1p7, and HCV ΔCE2p7, have been built by inserting a stop codon at the end of p7 whereas ΔCE2p7E1 has been built by inserting a stop codon at the end of E1.

By “retrovirus” is meant a virus whose genome consists of a RNA molecule and that comprises a reverse-transcriptase, i.e. a member of the Retroviridae family. Retroviruses are divided into Oncovirus, Lentivirus and Spumavirus. Preferably said retrovirus is an oncovirus, e.g. MLV, ALV, RSV, or MPMV, a lentivirus, e.g. HIV-1, HIV-2, SIV, EIAV, or CAEV, or a spumavirus such as HFV. Genomes of these retroviruses are readily available in databanks.

In the context of the invention “a nucleic sequence comprising a packaging competent retrovirus-derived genome” is intended for a sequence that comprises the retroviral nucleic acid sequences known as “cis-acting” sequences. These include the Long Terminal Repeats (LTRs) for the control of transcription and integration, the psi sequence necessary for encapsidation, and the Primer Binding site (PBS) and polypurine track (PPT) sequences necessary for reverse transcription of the retroviral genome. Advantageously, said nucleic acid sequence comprising a packaging competent retrovirus-derived genome further comprises a transgene.

Said retroviral genome may be replication-defective or replication-competent, in the absence of any trans-complementing function. A replication-competent genome would further comprise the gag, pol, and env retroviral genes. In a replication-defective genome, the viral genes gag, pol, and env are deleted. However, assembly of viral pseudo-particles may be achieved by providing in trans another vector that comprises gag, pol and env but that is defective for the “cis” sequences. Their expression allows the encapsidation of the transgene, excluding the genes necessary for the multiplication of the viral genome and for the formation of complete viral particles.

As used herein, the term “transgene” designates the gene that is expressed in the target cell upon infection by the particles of the invention.

Examples of transgenes include a gene encoding a molecule of therapeutic interest, a marker gene, a gene coding for an immune modulator, an antigen, or a suicide gene.

A “marker gene” denotes a gene whose expression is detectable. For instance marker gene expression can generate a detectable signal, such as a fluorescence emission, a chromogenic reaction, or confer a growth advantage to the cells wherein it is expressed (antibiotic resistance genes).

An “immune modulator” refers to the product of a gene that modifies the activity of the immune system of a subject in vivo. Examples of immune modulators include cytokines, (e.g. interleukins, interferons, or haematopoietic colony stimulating factors), chemokines, and the like. Expression of an immune modulator by transformed cells may change the cellular environment and alter differentiation of immune cells and thus modify the type and the strength of immune response elicited against a given antigen.

An “antigen” refers to a molecule, such as a peptide, a polypeptide or a protein, against which an immune response is sought. Said antigen may be for instance a tumor, a bacterial, a pathogenic, a proteic, or a viral antigen.

A “suicide gene” is meant for a gene whose expression in cells induces programmed-cell death (apoptosis) such as the conditional Herpes Simplex virus type I thymidine kinase gene.

The “core protein from a retrovirus” refers to proteins encoded by the gag and pol genes. The gag gene encodes a polyprotein which is further processed by the retroviral protease into structural proteins that comprise the core. The poi gene encodes the retroviral protease, reverse-transcriptase, and integrase.

A “pharmaceutically acceptable carrier” refers to any vehicle wherein the vaccine composition according to the invention may be formulated. It includes a saline solution such as phosphate buffer saline. In general, a diluent or carrier is selected on the basis of the mode and route of administration, and standard pharmaceutical practice.

In the context of the present application, “vaccination” is intended for prophylactic or therapeutical vaccination. “Therapeutical vaccination” is meant for vaccination of a patient with HCV infection.

According to the invention, the term “subject” or “patient” is meant for any mammal likely to be infected with a hepacivirus, in particular with HCV. Human, chimpanzee, tamarin, and mice, especially human liver-xenogratfed mice are examples of hosts for hepaciviruses, and in particular HCV.

### Production of Hepacivirus Pseudo-Particles

The inventors have generated infectious pseudo-particles that contain functional, and more particularly unmodified, hepacivirus glycoproteins, in particular HCV glycoproteins, assembled onto retroviral core particles. Hepacivirus (HCV) E1E2, and optionally p7, are expressed from a polyprotein containing the core (C) protein or a fragment thereof, in particular the carboxy-terminus of the C protein, which served as signal peptide for E1 or E2, and the E1 and/or E2 glycoproteins. More generally, hepacivirus E1E2 may be expressed from a polyprotein containing a signal peptide from a heterologous type I membrane protein.

The invention thus provides a method for producing hepacivirus-like particles ex vivo comprising the steps of:
providing a first nucleic acid sequence comprising a packaging competent retrovirus-derived genome;
providing a second nucleic acid sequence comprising a cDNA encoding the core proteins from said retrovirus;
providing a third nucleic acid sequence comprising a cDNA encoding a polyprotein comprising successively a signal peptide from a type I membrane protein, preferably a hepacivirus core protein, and a hepacivirus E1 protein and/or a hepacivirus E2 protein;
transfecting host cells with said nucleic acid sequences and maintaining the transfected cells in culture for sufficient time to allow expression of the cDNAs to produce structural proteins from hepacivirus and retrovirus; and allowing the structural proteins to form virus-like particles.

The invention further provides a method for producing hepacivirus-like particles in vivo, which method comprises the steps of:
providing a first nucleic acid sequence comprising a packaging competent retrovirus-derived genome;
providing a second nucleic acid sequence comprising a cDNA encoding the core proteins from said retrovirus;
providing a third nucleic acid sequence comprising a cDNA encoding a polyprotein comprising successively a signal peptide from a type I membrane protein, preferably a hepacivirus core protein, and a hepacivirus E1 protein and/or a hepacivirus E2 protein;
transfecting cells of a subject in vivo with said nucleic acid sequences, to allow expression of the cDNAs to produce structural proteins from hepacivirus and retrovirus; and to allow the structural proteins to form virus-like particles.

Another aspect of the invention is the use of three nucleic acid sequences for the preparation of a medicament useful as a vaccine against a hepacivirus infection, i.e. hepatitis, wherein the nucleic acid sequences are:
a first nucleic acid sequence comprising a packaging competent retroviral-derived genome;
a second nucleic acid sequence comprising a cDNA encoding core proteins from said retrovirus;
a third nucleic acid sequence comprising a cDNA encoding a polyprotein comprising successively a signal peptide from a type I membrane protein, preferably a hepacivirus core protein, and a hepacivirus E1 protein and/or a hepacivirus E2 protein;

and, when transferred into cells of a subject, the nucleic acid sequences allow the production of structural proteins from hepacivirus and retrovirus, wherein the structural proteins form virus-like particles that are immunogenic.

Preferably, said third nucleic acid sequence comprises a cDNA encoding a polyprotein that further comprises a hepacivirus p7 protein. Thus, preferably said polyprotein comprises successively a signal peptide from a type I membrane protein, preferably a hepacivirus core protein, a hepacivirus E1 protein and/or a hepacivirus E2 protein, and optionally a hepacivirus p7 protein.

According to a specific embodiment, said packaging competent retroviral genome and core proteins are derived from a retrovirus selected from the group consisting of MLV, ALV, RSV, MPMV, HIV-1, HIV-2, SIV, EIAV, CAEV, and HFV.

Advantageously, the packaging competent retroviral genome further comprises a marker gene or an immune modulator.

In the method of the invention, said polyprotein may comprise a hepacivirus core protein linked to a hepacivirus E1 protein, or a hepacivirus core protein linked to a hepacivirus E2 protein, or successively a hepacivirus core protein, a hepacivirus E1 protein and a hepacivirus E2 protein, or successively a hepacivirus core protein, a hepacivirus E2 protein and a hepacivirus E1 protein. Said polyprotein may further comprise successively a hepacivirus core protein, a hepacivirus E1 protein and a p7 protein, or successively a hepacivirus core protein, a hepacivirus E2 protein and a hepacivirus p7 protein, or successively hepacivirus core protein, a hepacivirus E1 protein, a hepacivirus E2 protein and a hepacivirus p7 protein, or successively a hepacivirus core protein, a hepacivirus E2 protein, a hepacivirus p7 protein, and a hepacivirus E1 protein.

According to an embodiment, E1 and/or E2, and optionally p7 protein, are native proteins. According to another embodiment, E1 and/or E2 glycoproteins, and optionally p7 protein, are mutated to obtain particles that may be useful for characterizing the glycoprotein determinants for hepacivirus infectivity.

Preferably, said E1 and E2 glycoproteins are both derived from a same hepacivirus strain. Preferably, said E1 or E2 glycoprotein and p7 protein are both derived from a same hepacivirus strain. Still preferably, said E1 and E2 glycoproteins, and p7 protein are derived from a same hepacivirus strain.

According to another embodiment said hepacivirus core protein is a carboxy terminus form (ΔC) of hepacivirus core protein, comprising the core protein signal peptide.

Preferably said hepacivirus is a hepatitis C virus (HCV).

The invention thus provides a method for producing hepatitis C virus (HCV)-like particles ex vivo comprising the steps of:
providing a first nucleic acid sequence comprising a packaging competent retrovirus-derived genome;
providing a second nucleic acid sequence comprising a cDNA encoding the core proteins from said retrovirus;
providing a third nucleic acid sequence comprising a cDNA encoding a polyprotein comprising successively a signal peptide from a type I membrane protein, preferably a HCV core protein, and a HCV E1 protein and/or a HCV E2 protein;
transfecting host cells with said nucleic acid sequences and maintaining the transfected cells in culture for sufficient time to allow expression of the cDNAs to produce structural proteins from hepatitis C virus and retrovirus; and allowing the structural proteins to form virus-like particles.

The invention further provides a method for producing hepatitis C-virus (HCV)-like particles in vivo, which method comprises the steps of:
providing a first nucleic acid sequence comprising a packaging competent retrovirus-derived genome;
providing a second nucleic acid sequence comprising a cDNA encoding the core proteins from said retrovirus;
providing a third nucleic acid sequence comprising a cDNA encoding a polyprotein comprising successively a signal peptide from a type I membrane protein, preferably a HCV core protein, and a HCV E1 protein and/or a HCV E2 protein;
transfecting cells of a subject in vivo with said nucleic acid sequences, to allow expression of the cDNAs to produce structural proteins from hepatitis C virus and retrovirus; and to allow the structural proteins to form virus-like particles.

Another aspect of the invention is the use of three nucleic acid sequences for the preparation of a medicament useful as a vaccine against hepatitis C, wherein the nucleic acid sequences are:
a first nucleic acid sequence comprising a packaging competent retroviral-derived genome;
a second nucleic acid sequence comprising a cDNA encoding core proteins from said retrovirus;
a third nucleic acid sequence comprising a cDNA encoding a polyprotein comprising successively a signal peptide from a type I membrane protein, preferably a HCV core protein, and a HCV E1 protein and/or a HCV E2 protein;

and, when transferred into cells of a subject, the nucleic acid sequences allow the production of structural proteins from hepatitis C virus and retrovirus, wherein the structural proteins form virus-like particles that are immunogenic.

According to a specific embodiment, said packaging competent retroviral genome and core proteins are derived from a retrovirus selected from the group consisting of MLV, ALV, RSV, MPMV, HIV-1, HIV-2, SIV, EIAV, CAEV, and HFV.

Advantageously, the packaging competent retroviral genome further comprises a marker gene or an immune modulator

Preferably, said third nucleic acid sequence comprises a cDNA encoding a polyprotein that further comprises a HCV p7 protein. Thus, preferably said polyprotein comprises successively a signal peptide from a type I membrane protein, preferably a HCV core protein, a HCV E1 protein and/or a HCV E2 protein, and optionally a HCV p7 protein.

An example of HCV E1E2 and retroviral expression constructs is shown in FIGS. 6A and 6B.

In the method of the invention, said polyprotein may comprise a HCV core protein linked to a HCV E1 protein, a HCV core protein linked to a HCV E2 protein, or successively a HCV core protein, a HCV E1 protein and a HCV E2 protein, or successively a HCV core protein, a HCV E2 protein and a HCV E1 protein. Said polyprotein may further comprise successively a HCV core protein, a HCV E1 protein and a p7 protein, or successively a HCV core protein, a HCV E2 protein and a HCV p7 protein, or successively HCV core protein, a HCV E1 protein, a HCV E2 protein and a HCV p7 protein, or successively a HCV core protein, a HCV E2 protein, a HCV p7 protein, and a HCV E1 protein.

According to an embodiment, HCV E1 and/or E2, and optionally HCV p7 protein, are native proteins. According to another embodiment, HCV E1 and/or E2 glycoproteins, and optionally HCV p7 protein, are mutated to obtain particles that may be useful for characterizing the glycoprotein determinants for HCV infectivity. A preferred mutant of E2 protein is the C-terminally truncated form of E2 protein, wherein the C-terminal Ala residue (amino acid 746 in SEQ ID N° 16) has been deleted. In another preferred mutant of E2 protein, the hypervariable region I (located in the N-terminus region, i.e. the first 27 amino acids of the E2 protein after the signal peptide, i.e. amino-acids 384-410 of SEQ ID N° 16) is deleted.

Preferably, said E1 and E2 glycoproteins are both derived from a same HCV strain. Preferably, said E1 or E2 glycoprotein, and p7 protein are both derived from a same HCV strain. Still preferably, said E1 and E2 glycoproteins, and p7 protein are derived from a same HCV strain.

According to another embodiment said HCV core protein is a carboxy terminus form (ΔC) of HCV core protein. In particular, said HCV core protein may comprise the last 21 amino acids of the carboxy-terminus of HCV core.

For the purpose of transfection, said first, second and third nucleic acid sequences may be carried on a same vector, or on two or three separated vectors.

In particular, plasmoviruses, adenoretroviruses and replicating pseudo-viruses are examples of vectors suitable for carrying the above-mentioned sequences. A plasmovirus vaccine consists in such a plasmid DNA preparation, that allow expression of hepacivirus pseudo-particles after administration in an patient in order to elicit a immune response against said hepacivirus. Administration of such a plasmovirus vaccine being achieved for preventive vaccination into people at risk for hepacivirus-induced disease or for therapeutic vaccination into hepacivirus-infected patients. Adenoretroviruses consist in an alternative way to provide the above-mentioned nucleic acid sequences encoding hepacivirus pseudo-particles. In this case, it is possible to design three independent adenoretroviruses, i.e. recombinant adenoviruses, that encode the three nucleic acid sequences mentioned above (retroviral core and genome and hepacivirus glycoproteins), or, alternatively, it is also possible to design a single adenoretrovirus, derived from “guttless” recombinant adenoviruses, that contains the different nucleic acid sequences. Such adenoretroviruses can be administered to patient as for plasmoviruses, in order to elicit an anti-hepacivirus immune response. Replicating pseudo-retroviruses are another alternative possibility to express all the above-mentioned nucleic acid sequences encoding the hepacivirus pseudo-particles. Such structures are in fact hepacivirus pseudo-particles whose genome is engineered to allow, following infection, its propagation into cells of an inoculated patient, thereby inducing the production of further replicating hepacivirus-pseudo-particles. In this case the genome of a retrovirus is modified so as to express the hepacivirus E1E2 glycoproteins in place of the retroviral Env gene (encoding the retroviral glycoproteins). The genes encoding the retroviral core proteins are left unchanged. Furthermore an additional gene, encoding a marker gene or an immunomodulator, for example, can be expressed from this genome.

In accordance with the present invention there may be employed conventional molecular biology, microbiology, and recombinant DNA techniques within the skill of the art. Such techniques are explained fully in the literature. See, e.g., Sambrook et al., 1989; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription and Translation [B. D. Hames & S. J. Higgins, eds. (1984)]; Animal Cell Culture [R. I. Freshney, ed. (1986)]; Immobilized Cells and Enzymes [IRL Press, (1986)]; B. Perbal, A Practical Guide To Molecular Cloning (1984); F. M. Ausubel et al., 1994.

In particular, the vectors of the invention may be introduced into the target cell by means of any technique known for the delivery of nucleic acids to the nucleus of cells, either in culture, ex vivo, or in vivo.

Introduction of the nucleic acid sequences may be performed by any standard method well known by one skilled in the art, e.g. transfection, electroporation, microinjection, transduction, cell fusion, DEAE dextran, calcium phosphate precipitation, or use of a gene gun (see for instance Wu et al., 1992; Wu et al, 1988).

The donor nucleic acid targeting system can also be introduced by lipofection. In certain embodiments, the use of liposomes and/or nanoparticles is contemplated for the introduction of the donor nucleic acid targeting system into host cells. Nanocapsules can generally entrap compounds in a stable and reproducible way. Ultrafine particles (sized around 0.1 μm) that can be designed using biodegradable polyalkyl-cyanoacrylate polymers are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The use of cationic lipids may promote encapsulation of negatively charged nucleic acids, and also promote fusion with negatively charged cell membranes (Feigner et al., 1989).

In vivo targeted gene delivery is described in international patent publication WO 95/28 494. Alternatively, the vector can be introduced in vivo by lipofection, using liposomes or nanoparticles as above described. It is also possible to introduce the vector in vivo using techniques that are similar to the techniques that are employed in vitro (e.g. transfection, electroporation . . . ).

### Transformed Cells

The invention further relates to a transformed host cell that contains:
a first nucleic acid sequence comprising a packaging competent retrovirus-derived genome;
a second nucleic acid sequence comprising a cDNA encoding the core proteins from said retrovirus; and
a third nucleic acid sequence comprising a cDNA encoding a polyprotein comprising successively a signal peptide from a type I membrane protein, preferably a hepacivirus core protein, and a hepacivirus E1 protein and/or a hepacivirus E2 protein.

Preferably, said third nucleic acid sequence comprises a cDNA encoding a polyprotein that further comprises a hepacivirus p7 protein. Thus, preferably said polyprotein comprises successively a signal peptide from a type I membrane protein, preferably a hepacivirus core protein, a hepacivirus E1 protein and/or a hepacivirus E2 protein, and optionally a hepacivirus p7 protein.

Such a transformed host cell is obtainable as described in a method above.

In another aspect, the invention relates to the use of a transformed host cell as defined above, for the identification of molecules capable of interfering with hepacivirus entry in cells. The invention provides in particular a method of ex vivo screening or identification of molecules capable of interfering with hepacivirus entry in cells comprising comparison of the level of transformed host cell fusion to a target host cell, in the presence or the absence of a candidate molecule. Said method preferably comprises the steps consisting of:
co-culturing a transformed host cell with a target host cell, in the absence or presence of a candidate molecule, under conditions that allow syncytia formation, i.e. cell-cell fusion, and hepacivirus-like particle entry in target host cell in the absence of any candidate molecule;
assessing syncytia formation in the absence and in the presence of said candidate molecule;
comparing syncytia formation measured in presence of said candidate molecule with syncytia formation measured in absence of any candidate molecule;
identifying as a molecule capable of interfering with hepacivirus entry the candidate molecule for which syncytia formation, as measured in the presence of said molecule, is decreased as compared to syncytia formation measured in the absence of any candidate molecule.

Preferably, said hepacivirus is a hepatitis C virus. The invention thus also relates to a transformed host cell that contains:
a first nucleic acid sequence comprising a packaging competent retrovirus-derived genome;
a second nucleic acid sequence comprising a cDNA encoding the core proteins from said retrovirus; and
a third nucleic acid sequence comprising a cDNA encoding a polyprotein comprising successively a signal peptide from a type I membrane protein, preferably a HCV core protein, and a HCV E1 protein and/or a HCV E2 protein.

According to another embodiment, said third nucleic acid sequence comprises a cDNA encoding a polyprotein that further comprises a HCV p7 protein. Preferably said polyprotein comprises successively a signal peptide from a type I membrane protein, preferably a HCV core protein, a HCV E1 protein and/or a HCV E2 protein, and optionally a HCV p7 protein.

Such a transformed host cell is obtainable as described in a method above.

In another aspect, the invention relates to the use of a transformed host cell as defined above, for the identification of molecules capable of interfering with HCV entry in cells. The invention provides in particular a method of ex vivo screening or identification of molecules capable of interfering with HCV entry in cells comprising comparison of the level of transformed host cell fusion to a target host cell, in the presence or the absence of a candidate molecule. Said method preferably comprises the steps consisting of:
co-culturing a transformed host cell with a target host cell, in the absence or presence of a candidate molecule, under conditions that allow syncytia formation, i.e. cell-cell fusion, and HCV-like particle entry in target host cell in the absence of any candidate molecule;
assessing syncytia formation in the absence and in the presence of said candidate molecule;
comparing syncytia formation measured in presence of said candidate molecule with syncytia formation measured in absence of any candidate molecule;
identifying as a molecule capable of interfering with HCV entry the candidate molecule for which syncytia formation, as measured in the presence of said molecule, is decreased as compared to syncytia formation measured in the absence of any candidate molecule.

Contacting a transformed host cell with a target host cell, and a candidate molecule can be carried out by contacting simultaneously said transformed host cell, target host cell and candidate molecule. Otherwise, two of these three elements can be contacted under conditions sufficient to allow their interaction before addition of the third missing element.

Preferably said target host cell is not transformed, i.e. said target host cell does not contains at least one of the first, second, and third nucleic acid sequence as defined above.

Syncytia formation can be readily assessed by one skilled in the art. Briefly, the coculture is submitted to a acidic pH drop by incubation for 5 min at pH-5 and incubated in a normal medium for an additional 12 hrs. Cultures are then stained by adding the May-Grunwald and Giemsa solutions (MERCK) according to the manufacturer recommendations. Cells containing two or more nuclei can be defined as syncytia. A fusion index is then defined as the percentage of (N−S)/T where N is the number of nuclei in the syncytia, S is the number of syncytia and T is the total number of nuclei counted.

### Hepacivirus-Like Particles

In the method described above no structural modifications of the E1E2 glycoproteins are required for their correct assembly on retroviral cores. The method of the invention thus makes it possible to generate high titre infectious hepacivirus pseudo-particles, and in particular HCV pseudo-particles, with functional E1E2 glycoproteins. As demonstrated herein, these particles constitute a valid model of hepacivirus virions, and in particular of HCV virions, as regards to early steps of viral infection cycle. However, depending of the intended use of the pseudo-particle of the invention, mutant E1, E2 or p7 protein may be assembled on the hepacivirus-like particle, with the limitations recited in the definition section.

The invention further relates to an infectious hepacivirus-like particle, comprising the core proteins from a retrovirus, E1 and/or E2 hepacivirus glycoprotein(s), and optionally hepacivirus p7 protein. Such a particle is obtainable by a method as described above.

According to an embodiment, the infectious particle of the invention may comprise native hepacivirus E1 protein, or native hepacivirus E2 protein, or native hepacivirus E1 protein and native hepacivirus E2 protein. Preferably said E1 and E2 glycoproteins are both derived from a same hepacivirus strain. According to another embodiment, E1 and/or E2 glycoproteins are mutated.

According to another embodiment the infectious particle of the invention may comprise native hepacivirus E1 and native hepacivirus p7 proteins, or native hepacivirus E2 and native hepacivirus p7 proteins, or native hepacivirus E1 protein, native hepacivirus E2 protein and native hepacivirus p7 protein Preferably, said E1 or E2 glycoprotein and p7 protein are both derived from a same hepacivirus strain. Still preferably, said E1 and E2 glycoproteins, and p7 protein are derived from a same hepacivirus strain. According to another embodiment, E1 and/or E2 glycoproteins and/or p7 protein are mutated.

Preferably, said hepacivirus is a hepatitis C virus. The invention thus also relates to an infectious HCV-like particle, comprising the core proteins from a retrovirus, E1 and/or E2 HCV glycoprotein(s), and optionally p7 protein. Such a particle is obtainable by a method as described above.

According to an embodiment, the infectious particle of the invention may comprise native HCV E1 protein, or native HCV E2 protein, or native HCV E1 protein and native HCV E2 protein. Preferably said E1 and E2 glycoproteins are both derived from a same HCV strain. According to another embodiment, E1 and/or E2 glycoproteins are mutated.

According to another embodiment the infectious particle of the invention may comprise native HCV E1 and native HCV p7 proteins, or native HCV E2 and native HCV p7 proteins, or native HCV E1 protein, native HCV E2 protein and native HCV p7 protein Preferably, said E1 or E2 glycoprotein and p7 protein are both derived from a same HCV strain. Still preferably, said E1 and E2 glycoproteins, and p7 protein are derived from a same HCV strain. According to another embodiment, E1 and/or E2 glycoproteins and/or p7 protein are mutated.

In the above embodiment, a mutated E2 protein is preferably the C-terminally truncated form of E2 protein, wherein the C-terminal Ala residue (amino acid 746 in SEQ ID N° 16) has been deleted.

In another preferred embodiment, the hypervariable region I (located in the N-terminus region, i.e. the first 27 amino acids of the E2 protein after the signal peptide, i.e. amino-acids 384-410 of SEQ ID N° 16) is deleted. The HCV-like particles (called ΔHVR1) produced with E2 protein deleted from this region are particularly advantageous as a diagnostic tool or in vaccination, to enhance induction or binding of neutralizing antibodies. The ΔHVR1 HCV-like particles are also of interest to identify the epitopes/mechanisms within the HCV glycoproteins that can be targeted/inhibited by neutralizing antibodies or other therapeutic agents.

Said retrovirus may be selected from the group consisting of MLV, ALV, RSV, MPMV, HIV-1, HIV-2, SIV, EIAV, CAEV, and HFV.

Advantageously, said infectious particles further carry a transgene. For instance said transgene may be a marker gene which make it possible to follow-up cell infection by the infectious particles of the invention and can find application for instance in the identification of a cell receptor involved in hepacivirus entry, and in particular HCV entry. Said transgene can also be a gene encoding a molecule of therapeutic interest and/or a suicide gene. Accordingly, the particles of the invention that specifically target primary or cancerous hepatocytes comprise a useful vector for gene transfer and/or gene therapy.

### Use of the Infectious Hepacivirus-Like Particles of the Invention

Hepacivirus Cell Receptor Identification

High infectivity of these particles makes it possible for the investigation of the role of hepacivirus (HCV) E1 and E2 glycoproteins and their potential receptors in cell entry, hepacivirus host-range and neutralisation by antibodies from hepacivirus patient sera. These particles make it possible for the investigation of the role of p7 protein in E1E2 maturation and functions, viral assembly, budding and release.

The invention therefore concerns the use of a hepacivirus-like infectious particle as described above, for ex vivo identification of a cell receptor for hepacivirus E1 and/or E2 glycoprotein.

According to an embodiment, the invention provides a method for ex vivo identification of a receptor for hepacivirus E1 and/or E2 glycoprotein comprising detection of the binding of said particle to a cell receptor. More specifically, the method may comprise the steps consisting of:
contacting a cell susceptible to hepacivirus infection with an infectious hepacivirus-like particle of the invention, under conditions sufficient to allow specific binding of said particle to a receptor expressed at the surface of said cell;
detecting binding of said particle to a receptor; and
identifying said receptor.

Preferably, said hepacivirus is a hepatitis C virus. The invention therefore concerns the use of an HCV-like infectious particle as described above, for ex vivo identification of a cell receptor for HCV E1 and/or E2 glycoprotein.

According to an embodiment, the invention provides a method for ex vivo identification of a receptor for HCV E1 and/or E2 glycoprotein comprising detection of the binding of said particle to a cell receptor. More specifically, the method may comprise the steps consisting of:
contacting a cell susceptible to HCV infection with an infectious HCV-like particle of the invention, under conditions sufficient to allow specific binding of said particle to a receptor expressed at the surface of said cell;
detecting binding of said particle to a receptor; and
identifying said receptor.

A cell susceptible to a hepacivirus infection, and in particular to a HCV infection, may preferably be selected from the group consisting of a hepatocyte cell line, such as Huh-7 human hepatocellular carcinoma (Nakabayashi et al., 1982), PLC/PRF/5 human hepatoma (CRL-8024), Hep3B human hepatocellular carcinoma (ATCC HB-8064), or HepG2 human hepatocellular carcinoma (HB-8065), and a primary human hepatocyte. Primary human hepatocytes may be isolated from human adult biopsy samples according to procedures well-known by one skilled in the art. One can for example refer to Guguen-Guillouzo and Guillouzo (1986). Otherwise such cells are commercially available, and can be purchased for instance from Biopredic International (Rennes, France).

Detection of particle binding to a receptor can be achieved according to classical procedures well known by one skilled in the art. For instance, this could involve radioactive, enzyme or fluorescent labelling of the particles of the invention, and subsequent detection with an appropriate method. A number of fluorescent materials are known and can be utilized as labels. These include, for example, fluorescein, rhodamine, auramine, Texas Red. Enzyme labels consist in conjugation of an enzyme to a molecule of interest, e.g. a polypeptide, and can be detected by any of colorimetric, spectrophotometric, or fluorospectrophotometric techniques. Flow cytometry analysis (FAGS) together with labelled antibodies directed against E1 or E2 proteins harboured by the pseudo-particles of the invention is also appropriate.

According to another embodiment, the invention provides a method for ex vivo identifying a cell receptor for a hepacivirus comprising the step consisting of:
transfecting a cell which is not permissive for hepacivirus infection with a nucleic acid sequence encoding a protein likely to be a receptor for hepacivirus;
contacting said transformed cell with a hepacivirus-like particle of the invention;
determining whether said transformed cell has become permissive or not for hepacivirus infection; and
identifying as a cell receptor for a hepacivirus said protein expressed by the transformed cell that has become permissive.

Preferably, the invention provides a method for ex vivo identifying a cell receptor for HCV comprising the step consisting of:
transfecting a cell which is not permissive for HCV infection with a nucleic acid sequence encoding a protein likely to be a receptor for HCV;
contacting said transformed cell with a HCV-like particle of the invention;
determining whether said transformed cell has become permissive or not for HCV infection; and
identifying as a cell receptor for HCV said protein expressed by the transformed cell that has become permissive.

Determination of whether the transformed cell has become permissive for hepacivirus infection, and in particular HCV infection, can be readily achieved using the hepacivirus-like (HCV-like) particles of the invention. In particular, where said particles carry a marker gene, such as GFP, permissivity (i.e. the capacity of cells to be infected with a hepacivirus, or with a hepacivirus-like particle, in particular with HCV or with HCV-like particles) can be assessed by FACS analysis of the transformed cells. Where the marker gene is an antibiotic resistance gene, identification of cells infected by the hepacivirus-like (HCV-like) particle is readily achieved through exposure to said antibiotic.

Where one does not suspect a given protein to be a receptor for hepacivirus entry, in cells, the above method can advantageously be adapted for the screening and the identification of a cell receptor for a hepacivirus, such as HCV. In particular, an expression cDNA library can be prepared, for instance from a cDNA library obtained by reverse-transcription of cellular mRNAs from a cell permissive for hepacivirus infection, in particular for HCV infection. Expression of such a cDNA library would be driven by a constitutive promoter whose nucleic acid sequence has been fused to the cDNA library in suitable vectors. Such a library would contain a vector encoding a cell receptor for a hepacivirus, for instance for HCV. Non permissive cells can then be transfected with this expression library and further screened for the identification of a cell receptor for a hepacivirus.

To this end, the invention proposes a method for ex vivo identifying a cell receptor for hepacivirus comprising the step consisting of:
providing an expression cDNA library obtained from a cell permissive for hepacivirus infection;
transfecting cells that are not permissive for hepacivirus infection with said expression cDNA library;
contacting said transformed cells with hepacivirus-like particles of the invention;
identifying and isolating those transformed cells that have become permissive for hepacivirus infection;
isolating the expression vector transfected in cells that have become permissive; and
identifying as a receptor for hepacivirus the proteins encoded by the cDNA sequence of said isolated expression vectors.

Preferably said hepacivirus is a hepatitis C virus. The invention thus proposes a method for ex vivo identifying a cell receptor for HCV comprising the step consisting of:
providing an expression cDNA library obtained from a cell permissive for HCV infection;
transfecting cells that are not permissive for HCV infection with said expression cDNA library;
contacting said transformed cells with HCV-like particles of the invention;
identifying and isolating those transformed cells that have become permissive for HCV infection;
isolating the expression vector transfected in cells that have become permissive; and
identifying as a receptor for HCV the proteins encoded by the cDNA sequence of said isolated expression vectors.

Determination of whether the transformed cell has become permissive for hepacivirus (HCV) infection can be readily achieved using the hepacivirus-like (HCV-like) particles of the invention. In particular, where said particles carry a marker gene, such as GFP, permissivity (i.e. the capacity of cells to be infected with hepacivirus, and in particular with hepacivirus-like particles, for instance with HCV or with HCV-like particles) can be assessed by FACS analysis of the transformed cells. Where the marker gene is an antibiotic resistance gene, identification of cells infected by hepacivirus-like particles, in particular by HCV-like particles, is readily achieved through exposure to said antibiotic.

Advantageously, the expression cDNA library is expressed from retroviral vectors that comprise glycoproteins that allow infection of the hepacivirus (HCV) non permissive cells. Such glycoproteins can be the VSV-G glycoprotein derived from vesicular stomatitis virus (VSV) whose receptor is expressed in most cell types ex vivo. Such viral particles can be assembled using a packaging competent retrovirus-derived genome that comprises the expression cDNA library, and optionally a marker gene. According to this embodiment the method for isolating the expression vector expressed in cells that have become permissive to infection by the hepacivirus-like (HCV-like) particles of the invention is greatly facilitated. Indeed this latter embodiment is particularly advantageous in that the process of cell infection with retroviral vectors has greater efficacy, as compared to cell transfection. Furthermore, cell infection leads to stable integration of viral genome in the cellular genome, which greatly facilitates recovery and isolation of the cDNA of interest. Accordingly, transgenes, i.e. cDNA and marker gene that are carried by the pseudo-particles of the invention, are found to be stably expressed by infected cells. This in contrast with classical vectors used for transfection that do not integrate into cellular genome and for which expression may be transient.

Identification of Agents Interfering with Hepacivirus Infection

In another aspect, the invention relates to the use of an infectious particle as defined above, for the identification of molecules capable of interfering with hepacivirus, and in particular HCV, entry in cells.

In particular, herein is provided a method of ex vivo screening or identification of molecules capable of interfering with hepacivirus entry in cells comprising comparison of the level of cell infection by the particles of the invention in the presence or the absence of a candidate molecule. Said method preferably comprises the steps consisting of:
contacting a cell susceptible to hepacivirus infection with an infectious hepacivirus-like particle, in the absence or presence of a candidate molecule, under conditions that allow cell infection with hepacivirus-like particle in the absence of any candidate molecule;
assessing cell infectivity in the absence and in the presence of said candidate molecule;
comparing cell infectivity measured in presence of said candidate molecule with cell infectivity measured in absence of any candidate molecule;
identifying as a molecule capable of interfering with hepacivirus entry the candidate molecule for which cell infectivity, as measured in the presence of said molecule, is decreased as compared to cell infectivity measured in the absence of any candidate molecule.

Contacting a cell susceptible to hepacivirus infection with an infectious hepacivirus-like particle, and a candidate molecule can be carried out by contacting simultaneously said cell, hepacivirus-like particle and candidate molecule. Otherwise, two of these three elements can be contacted under conditions sufficient to allow their interaction before addition of the third missing element.

Cell infectivity can be readily assessed by one skilled in the art. One can take advantage of the embodiment wherein the infectious hepacivirus-like particle carries a detectable marker gene to detect cell infection. In a preferred embodiment, the marker gene is a fluorescent marker gene, such as GFP, and the infection is detected by means of fluorescence measurement, for instance by flow cytometry analysis of cells contacted with said infectious particles.

A cell suitable to be used in the method of identification of molecules interfering with hepacivirus cell entry may be selected from the group consisting of a hepatocyte cell line and a primary human hepatocyte, as described above.

Preferably said hepacivirus is a hepatitis C virus. The invention thus further provides a method of ex vivo screening or identification of molecules capable of interfering with HCV entry in cells comprising comparison of the level of cell infection by the particles of the invention in the presence or the absence of a candidate molecule. Said method preferably comprises the steps consisting of:
contacting a cell susceptible to HCV infection with an infectious HCV-like particle, in the absence or presence of a candidate molecule, under conditions that allow cell infection with HCV-like particle in the absence of any candidate molecule;
assessing cell infectivity in the absence and in the presence of said candidate molecule;
comparing cell infectivity measured in presence of said candidate molecule with cell infectivity measured in absence of any candidate molecule;
identifying as a molecule capable of interfering with HCV entry the candidate molecule for which cell infectivity, as measured in the presence of said molecule, is decreased as compared to cell infectivity measured in the absence of any candidate molecule.

Contacting a cell susceptible to HCV infection with an infectious HCV-like particle, and a candidate molecule can be carried out by contacting simultaneously said cell, HCV-like particle and candidate molecule. Otherwise, two of these three elements can be contacted under conditions sufficient to allow their interaction before addition of the third missing element.

Cell infectivity can be readily assessed by one skilled in the art. One can take advantage of the embodiment wherein the infectious HCV-like particle carries a detectable marker gene to detect cell infection. In a preferred embodiment, the marker gene is a fluorescent marker gene, such as GFP, and the infection is detected by means of fluorescence measurement, for instance by flow cytometry analysis of cells contacted with said infectious particles.

A cell suitable to be used in the method of identification of molecules interfering with HCV cell entry may be selected from the group consisting of a hepatocyte cell line and a primary human hepatocyte, as described above.

Such molecules' capable of interfering with hepacivirus, and in particular with HCV, entry in cells may constitute new antiviral drugs.

Hepacivirus Infection Diagnosis

The infectious particles of the invention are further useful for diagnosis of hepacivirus infection and follow-up of hepacivirus infection, for instance to assess efficacy of a therapy in a patient.

The invention thus concerns the use of an infectious hepacivirus-like particle for the in vitro detection of antibodies directed against hepacivirus in a biological sample from a subject susceptible to be infected with hepacivirus. Said biological sample may be a biological fluid, such as blood or serum, or a tissue biopsy. In a specific embodiment, said antibodies are directed against E1 and/or E2 hepacivirus glycoproteins.

Accordingly, the invention provides a method of in vitro diagnosis of a hepacivirus infection in a patient comprising detecting immune complexes formed by interaction of anti-hepacivirus antibodies likely to be present in a biological sample of the patient, with hepacivirus-like particle of the invention. Said method may in particular comprise the steps consisting of:
contacting a biological sample with an infectious hepacivirus-like particle of the invention under conditions sufficient to allow formation of complexes by binding of said infectious particle to antibodies directed against hepacivirus present in the biological sample;
detecting said complexes, which presence is indicative of a hepacivirus infection.

The presence of antibodies reactive with hepacivirus-like particles can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the hepacivirus-like particle and the antibody or antibodies reacted therewith.

In another embodiment, said method of in vitro diagnosis of a hepacivirus infection in a patient comprises detecting an inhibitory effect of anti-hepacivirus antibodies likely to be present in a biological sample of the patient, on the infection of a permissive cell by a hepacivirus-like particle of the invention. Said method may in particular comprise the steps consisting of:
contacting a cell permissive for hepacivirus infection with a hepacivirus-like particle and a biological sample;
comparing cell infectivity measured in presence of said biological sample with cell infectivity measured in absence of said biological sample;
detecting the inhibition of hepacivirus-like particle infection of a permissive cell as a decrease in cell infectivity measured in presence of said biological sample compared with cell infectivity measured in absence of said biological sample, said inhibition being indicative of a hepacivirus infection.

This embodiment is advantageous in that the method relies on the detection of the specific antibodies that are neutralizing for cell infection, i.e. those patient's antibodies that are effective against viraemia.

In a further embodiment of this invention, commercial diagnostic kits may be useful to carry out the above diagnosis methods, by detecting the presence or absence of immune complexes formed by hepacivirus particles and antibodies directed against hepacivirus in a biological sample from a subject susceptible to be infected with hepacivirus, or by detecting an inhibition of hepacivirus-like particle infection of a permissive cell by anti-hepacivirus neutralizing antibodies likely to be present in a biological sample of the patient. Such kits may comprise at least a hepacivirus-like particle of the present invention. Where the method involves detection of immune complexes, the kits may further comprise appropriate means of detection of said immune complexes. Preferably the kit of the invention further comprises directions, and protocols, depending upon the method selected, e.g., “competitive”, “sandwich”, and the like. The kits may also contain peripheral reagents such as buffers, stabilizers, etc.

Preferably said hepacivirus is a hepatitis C virus. The infectious particles of the invention are further useful for diagnosis of HCV infection and follow-up of HCV infection, for instance to assess efficacy of a therapy in a patient.

The invention thus concerns the use of an infectious HCV-like particle for the in vitro detection of antibodies directed against HCV in a biological sample from a subject susceptible to be infected with HCV. Said biological sample may be a biological fluid, such as blood or serum, or a tissue biopsy. In a specific embodiment, said antibodies are directed against E1 and/or E2 HCV glycoproteins.

Accordingly, the invention provides a method of in vitro diagnosis of a HCV infection in a patient comprising detecting immune complexes formed by interaction of anti-HCV antibodies likely to be present in a biological sample of the patient, with HCV-like particle of the invention. Said method may in particular comprise the steps consisting of:
contacting a biological sample with an infectious HCV-like particle of the invention under conditions sufficient to allow formation of complexes by binding of said infectious particle to antibodies directed against HCV present in the biological sample;
detecting said complexes, which presence is indicative of a HCV infection.

The presence of antibodies reactive with HCV-like particles can be detected using standard electrophoretic and immunodiagnostic techniques, including immunoassays such as competition, direct reaction, or sandwich type assays, as described above. The reactions generally include revealing labels such as fluorescent, chemiluminescent, radioactive, enzymatic labels or dye molecules, or other methods for detecting the formation of a complex between the HCV-like particle and the antibody or antibodies reacted therewith.

In another embodiment, said method of in vitro diagnosis of a HCV infection in a patient comprises detecting an inhibitory effect of anti-HCV antibodies likely to be present in a biological sample of the patient, on the infection of a permissive cell by a HCV-like particle of the invention. Said method may in particular comprise the steps consisting of:
contacting a cell permissive for HCV infection with a HCV-like particle and a biological sample;
comparing cell infectivity measured in presence of said biological sample with cell infectivity measured in absence of said biological sample;
detecting the inhibition of HCV-like particle infection of a permissive cell as a decrease in cell infectivity measured in presence of said biological sample compared with cell infectivity measured in absence of said biological sample, said inhibition being indicative of a HCV infection.

This embodiment is advantageous in that the method relies on the detection of the specific antibodies that are neutralizing for cell infection, i.e. those patient's antibodies that are effective against viraemia.

In a further embodiment of this invention, commercial diagnostic kits may be useful to carry out the above diagnosis methods, by detecting the presence or absence of immune complexes formed by HCV particles and antibodies directed against HCV in a biological sample from a subject susceptible to be infected with HCV, or by detecting an inhibition of HCV-like particle infection of a permissive cell by anti-HCV neutralizing antibodies likely to be present in a biological sample of the patient. Such kits may comprise at least a HCV-like particle of the present invention. Where the method involves detection of immune complexes, the kits may further comprise appropriate means of detection of said immune complexes. Preferably the kit of the invention further comprises directions, and protocols, depending upon the method selected, e.g., “competitive”, “sandwich”, and the like. The kits may also contain peripheral reagents such as buffers, stabilizers, etc.

Vaccination Against Hepacivirus Infection

In another aspect of the invention, the infectious hepacivirus-like particles may be used for vaccination purposes.

According to an embodiment, the invention thus proposes a method of vaccination, notably against hepacivirus infection, that comprises administration of a hepacivirus-like particle to a subject in need thereof. The invention also relates to a vaccine composition comprising a hepacivirus-like particle and a pharmaceutically acceptable carrier. The invention further provides a immunogenic composition comprising in a pharmaceutical acceptable carrier, a hepacivirus-like particle disclosed herein.

The vaccine and immunogenic compositions of the invention may be drawn to confer immunity, or elicit an immune response against hepacivirus.

However, where the hepacivirus-like particles of the invention further carry an additional gene encoding another antigen, different from hepacivirus antigens, the invention provides a recombinant viral vaccine useful to raise an immune response against said antigen. Actually, the use of pseudo-particles described herein makes it possible to improve the elicited immune response through combining several presentations and processing pathways of an antigen. For instance, a vaccine composition of the invention, when administered, results in the hepacivirus-like particles infecting cells of the host. The transgene encoding the antigen is then integrated in the cellular genome, and subsequently expressed by the cell, such that there is both a cellular and a humoral immune response elicited by the vaccine composition.

Advantageously, the hepacivirus-like particles may further carry a transgene encoding an immune modulator, which allows for enhancement of the raised immune reaction.

Preferably said hepacivirus is a hepatitis C virus. The invention thus proposes a method of vaccination, notably against HCV infection, that comprises administration of a HCV-like particle to a subject in need thereof. The invention also relates to a vaccine composition comprising a HCV-like particle and a pharmaceutically acceptable carrier. The invention further provides a immunogenic composition comprising in a pharmaceutical acceptable carrier, a HCV-like particle disclosed herein.

The vaccine and immunogenic compositions of the invention may be drawn to confer immunity, or elicit an immune response against HCV.

However, where the HCV-like particles of the invention further carry an additional gene encoding another antigen, different from HCV antigens, the invention provides a recombinant viral vaccine useful to raise an immune response against said antigen. Actually, the use of pseudo-particles described herein makes it possible to improve the elicited immune response through combining several presentations and processing pathways of an antigen. For instance, a vaccine composition of the invention, when administered, results in the HCV-like particles infecting cells of the host. The transgene encoding the antigen is then integrated in the cellular genome, and subsequently expressed by the cell, such that there is both a cellular and a humoral immune response elicited by the vaccine composition.

Advantageously, the HCV-like particles may further carry a transgene encoding an immune modulator, which allows for enhancement of the raised immune reaction.

The vaccination or immunogenic composition of the present invention may additionally contain an adjuvant. A number of adjuvants are known to those skilled in the art. Examples of suitable adjuvants include, for example, include aluminum hydroxide; Saponin; detergents such as Tween 80; animal, mineral or vegetable oils, Corynebacterium or Propionibacterium-derived adjuvants; Mycobacterium bovis (Bacillus Calmette and Guerinn, or BCG); cytokines; acrylic acid polymers such as carbomer; EMA; or combinations thereof.

The route of administration is any conventional route used in the vaccine field. As general guidance, a vaccine composition of the invention is administered via a mucosal surface, e.g., an ocular, intranasal, pulmonary, oral, intestinal, rectal, vaginal, and urinary tract surface; or via a parenteral route, e.g., by an intravenous, subcutaneous, intraperitoneat, intradermal, intraepidermal, or intramuscular route. The choice of administration route depends on the formulation that is selected.

Vectors for Gene Transfer and/or Therapy

In still another embodiment the particles of the invention may be used as vectors for gene transfer and/or gene therapy. Gene therapy is defined as the introduction of genetic material into a cell in order to either change its phenotype or genotype. Owing to their tropism for hepatic cells, either primary or cancerous, the hepacivirus-like particles, and in particular the HCV-like particles, described herein comprise an efficient gene delivery system specific for hepatocytes. Furthermore, such a delivery system is amenable to scale up for reproducibly producing large titers of infectious, replication-defective hepacivirus-like particles, in particular HCV-like particles.

Accordingly, the invention relates to a method for in vivo or in vitro transferring a transgene of interest in a cell, which method comprises infecting a cell with a hepacivirus-like particle of the invention, wherein the particle carries a transgene of interest.

The invention further relates to the use of a hepacivirus-like particle of the invention, that carries a transgene of interest, for the preparation of a medicament for the prevention or treatment of a disease in a patient, wherein the hepacivirus-like particle allows the transfer of the transgene of interest into a cell of the patient, and encodes a product that has a prophylactic or therapeutic effect against the disease.

Preferably said hepacivirus is a hepatitis C virus. The invention thus proposes a method for in vivo or in vitro transferring a transgene of interest in a cell, which method comprises infecting a cell with a HCV-like particle of the invention, wherein the particle carries a transgene of interest.

The invention further relates to the use of a HCV-like particle of the invention, that carries a transgene of interest, for the preparation of a medicament for the prevention or treatment of a disease in a patient, wherein the HCV-like particle allows the transfer of the transgene of interest into a cell of the patient, and encodes a product that has a prophylactic or therapeutic effect against the disease.

Preferably, the targeted cell is a hepatic cell.

The invention will be further understood in view of the following examples and the annexed figures.

### LEGEND TO THE FIGURES

FIG. 1 depicts the results of infectivity experiments performed with HCV pseudo-particles of 1a genotype and different target cell types. Results are displayed as transducing units (TU) per ml of supernatant (mean±standard deviations of up to six experiments) for HCVpp. The infectivity on Huh-7 cells of HCVpp concentrated 100 times by ultra-centrifugation is shown (100×).

FIG. 2 represents the infectious titres determined on Huh-7 target cells with HCV pseudo-particles generated without E1E2 (lane a), without retroviral core proteins (lane b), with MLV-G2A assembly-defective core proteins (lane c), with HIV-1 core proteins (lane d), with E1 or E2 alone (lane e or f, respectively), with E1+E2 expressed in trans from two independent vectors (lane g), with HCV-1a E1E2 expressed from the same vector in cis (lane h), or with HCV-1 b E1E2 (lane i). HCVpp were treated with 25 μM AZT (3′-azido-3′-deoxythymidine; Sigma-Aldrich, France) before and during infection of target cells (lane j). Infectivity of HCV pseudo-particles generated with E1 from 1a HCV genotype and E2 form 1b HCV or genotype (lane k) or with E1 from 1 b HCV genotype and E2 form 1a HCV or genotype (lane l). Results are expressed as TU/ml and are displayed as mean±standard deviations of up to four experiments.

FIG. 3 shows the results of infection on human primary hepatocytes derived from two donors with of HCVpp of genotype 1a. Infectivity is expressed as percentage of infectivity determined on Huh-7 cells.

FIG. 4 displays neutralisation of HCV pseudo-particles with monoclonal antibodies against E1 (A4) or E2 (H31, H33, H35, H44, H48, H53, H54, H60 and H61) glycoproteins of genotype 1a, with pooled antibodies (Hmix), with no antibodies or using pseudo-particles generated with VSV-G (control). Neutralization of the control was achieved with the VSV-G neutralising 41A.1 monoclonal antibody. Results are expressed as the percentages of inhibition of the average infectious titres±standard deviations relative to incubation in the absence of antibodies.

FIG. 5 represents neutralisation of HCVpp with HCV patient sera. The genotype of HCV diagnosed in these patients is indicated in brackets. Results are expressed as percentages of inhibition of the average infectious titres±standard deviations relative to incubation with control sera from healthy individuals.

FIGS. 6A and 6B show HCV E1E2 and retroviral expression constructs. (A): A cDNA derived from the HCV polyprotein gene was used to express the E1E2 glycoproteins and the carboxy-terminus of the C protein, which provides the signal peptide for E1 (SP E1). The position of stop codons (star) inserted in the expression constructs to terminate translation of the proteins is shown. The transmembrane domain (TMD) of E1 provides the signal peptide (SP E2) for the E2 glycoprotein. (B): The expression constructs encoding the different components required to assemble infectious pseudo-particles are shown. The filled boxes represent the viral genes and the marker gene (GFP) transferred to the infected cells. The open boxes show the cis-acting sequences. LTR, long terminal repeat; CMV, human cytomegalovirus immediate-early promoter; PBS, primer binding site; Ψ, packaging sequence; PPT, poly-purine track; polyA, polyadenylation site; SD, splice donor site; SA, splice acceptor site. Vector particles were produced by cotransfection of plasmids harboring the packaging functions, the transfer vector and the viral glycoproteins into 293 T cells. The viral glycoproteins were the HCV E1 and E2 glycoproteins, expressed individually or as a CE1E2, the VSV-G or the RD114 glycoproteins. The supernatants of transfected cells were collected during transient expression, concentrated by ultracentrifugation, and used for target cell transduction.

FIG. 7 shows incorporation of ΔHVR1 HCV pseudo-particles. Immunoblots of lysates of 293 T transfected cells and of pseudo-particles pelleted through 20% sucrose-cushions are shown. The positions of the molecular weight markers are shown (kDa).

FIG. 8 represents the infectivity of ΔHVR1 HCV pseudo-particles. Supernatants from producer cells were diluted and used to infect Huh-7 target cells before assessing transduction efficiency three days post-infection by FACS analysis.

### EXAMPLES

### Example 1

### Generation of HCV Pseudo-Particles (HCVpp)

HCV pseudo-particles (HCVpp) were generated by assembling the full-length, unmodified E1 glycoprotein and the C-terminally truncated form of E2 protein (deletion of the C-terminal Ala 746 residue, SEQ ID N° 16) onto retroviral core proteins derived from murine leukemia virus (MLV). To investigate further whether functional HCVpp could also be produced with E1 and E2 expressed in trans or with only one of the two glycoproteins, expression vectors that encoded individually either E1 or E2 glycoproteins were designed.

Construction of Expression Vectors Encoding the Viral Components, i.e., E1, E2, or E1E2 Glycoproteins and Viral Core Proteins

Plasmids expressing wild type E1E2 polyproteins were constructed by standard methods (Sambrook et al., 1989).

Briefly, the phCMV-7a expression vector encoding the E1 and E2 glycoproteins from a 1a type HCV was generated by inserting the blunted ClaI and StuI restriction fragment encoding the last 60 residues of HCV core (C) and all of E1 and E2 proteins from the pTM1p5E1E2(745) vector (Op De Beeck et al., 2000) into the BamHI digested and Klenow blunted vector phCMV-G (Nègre et al., 2000).

The phCMV-E1 expression vector, expressing only HCV E1 glycoprotein, was derived from phCMV-7a by adding a stop codon to the C-terminus of E1 with primers 5′-actaaacgacgcaaagctgc (SEQ ID N° 2) and 5′-cgcggatcctacgcgtcgacgccggcaaa (SEQ ID N° 3). The resulting PCR fragment was digested with BamHI and ligated into BamHI-digested phCMV-7a.

phCMV-E2, expressing only HCV E2 glycoprotein, was obtained by fusing the N-terminus of E2 to the C-terminus of the HCV core using two PCR fragments generated with the two primer pairs 5′-tgcccgcttcagccgaaacccacgtcaccggggga (SEQ ID N° 4)+5′-gccagaagtcagatgctcaagg (SEQ ID N° 5) and 5′-tactctgagtccaaaccg (SEQ ID N° 6)+5′-gtgacgtgggtttcggctgaagcgggcacagtcag (SEQ ID N° 7). The two PCR fragments were then fused in a second round PCR. The resulting DNA fragment was digested with BamHI and ligated into BamHI cut phCMV-7a.

The sequence of the phCMV-ΔC E1 vector is shown in SEQ ID No 8, whereas the aminoacid sequence of E1 protein is shown in SEQ ID No 9. The sequence of the phCMV-ΔC E1E2 vector is shown in SEQ ID No 10, whereas the aminoacid sequence if the E1E2 polyprotein is shown in SEQ ID No 11. The sequence of the phCMV-ΔC E2 vector is shown in SEQ ID No 12, whereas the aminoacid sequence of E2 protein is shown in SEQ ID No 13.

Expression vectors for E1E2 glycoproteins of 1 b genotype were constructed by similar strategies.

HCV E1E2 were therefore expressed from a polyprotein containing the carboxy-terminus of the core (ΔC) protein, which served as signal peptide for E1, E2 or E1 and E2 glycoproteins.

Generation of HCV Pseudo-Particles

Retroviruses were chosen as platforms for assembly of HCVpp because their cores can incorporate a variety of different cellular and viral glycoproteins and because they can easily package and integrate genetic markers into host cell DNA.

HCVpp were produced by transfecting 293T human embryo kidney cells (ATCC CRL-1573) with three expression vectors encoding a ΔCE1E2, ΔCE1 or ΔCE2 polyprotein, the MLV core proteins and a packaging-competent MLV-derived genome harbouring the GFP (green fluorescent protein) marker gene. This construct contains the GFP marker gene, whose expression is driven by the CMV (cyto-megalovirus) immediate early promoter. Both CMV and GFP nucleic acid sequences where inserted in a retroviral vector derived from MLV in which the gag, pol and env viral gene where removed and in which the retroviral cis-acting elements that control vector genome packaging, reverse transcription and integration were retained.

Control pseudo-particles were generated with the VSV-G glycoprotein (Nègre et al., 2000), the RD114 virus envelope glycoprotein (Sandrin et al., 2002), and/or with assembly-defective MLV core proteins (MLV-G2A) (Swanstrom et al., 1997).

Briefly, expression constructs were transfected into 2.5×10⁶ 293 T cells seeded the day before in 10 cm plates using a calcium-phosphate transfection protocol (Clontech, France) according to the manufacturer's recommendations. The medium (8 ml/plate) was replaced 16 hrs after transfection. Supernatants containing the pseudo-particles were harvested 24 hrs later, filtered through 0.45 μm-pore-sized membranes and processed as described before (Nègre et al., 2000).

Immunoblot Analysis of Structural Components of the Pseudo Particles

Lysates of transfected cells and of pseudo-particles pelleted through 20% sucrose-cushions were immunobloted. Expression of E1 and E2 glycoproteins from HCV-1a genotype and of MLV core proteins was revealed in reducing and denaturing conditions with monoclonal antibodies against E1 (A4) (Dubuisson et al., 1994) and E2 (H52) (Flint et al., 1999) or with an anti-capsid (MLV CA) antiserum (ViroMed Biosafety Laboratories, USA), as described previously (Sandrin et al., 2002). VSV-G expressed in control pseudo-particles was detected with the monoclonal antibody P5D4 (Sigma-Aldrich, France).

Analysis of immunoblots of transfected cells showed that the structural components of the pseudo-particles were readily detected at the expected molecular weights; i.e. 30 kDa for E1, 60 kDa for E2, 60 kDa for VSV-G and 70 kDa for the RD114 glycoprotein. MLV core proteins were detected as a Gag protein precusor of 65 kDa that was partially processed by the MLV protease into mature core components.

The E1 and E2 glycoproteins were readily detected in the pellets of purified virions generated with the wild-type MLV core particles but not with the viral assembly-deficient MLV-G2A mutant. E2 present within the viral pellets migrated slightly slower than the cell-associated forms, due to modifications of the associated glycans by Golgi enzymes.

The presence of VSV-G in viral pellets generated with MLV-G2A assembly-defective core proteins is due to empty vesicles formed by VSV-G itself (Nègre et al., 2000).

Comparison of the relative levels of VSV-G or HCV glycoproteins in producer cell lysates versus viral pellets suggests efficient incorporation of E1 and E2 into viral particles. Likewise, could E1 and E2 glycoproteins efficiently assemble on retroviral core proteins derived from HIV-1, raising the possibility of pathogenic interactions between the two parental viruses, in vivo, as co-infection of patients with HCV and HIV is prevalent (Dieterich, 2002).

Altogether, these results indicate that transient over-expression of E1 and E2 in 293T cells leads to specific and efficient incorporation of HCV glycoproteins into pseudo-particles generated with retroviral cores. Since HCV envelope glycoproteins have been shown to be retained in the ER (Op De Beeck et al. 2001), this implies that HCVpp form by budding into the ER lumen or, alternatively, that saturation/leakiness of the ER retention allows a fraction of E1E2 to reach the cell surface where MLV budding normally occurs (Swanstrom and Wills, 1997).

Individual expression or co-expression in trans of E1 and E2, from distinct expression units, led to normal levels of synthesis, as compared to expression of both glycoproteins in cis, from a single E1E2 polyprotein. Moreover, HCVpp were found to incorporate similar levels of E2 glycoprotein, whether it was expressed alone, or co-expressed in cis or in trans with E1. Finally, incorporation of E1 expressed alone or co-expressed in trans, with E2, occurred but at reduced levels, consistent with the chaperone activity of E2 (Op De Beeck et al., 2001). Formation of HCVpp carrying E1 or E2 only will allow investigation of their respective roles in HCV cell entry and infectivity.

### Example 2

### HCVpp Cell Line Infectivity

Infectivity of HCVpp was assessed on a panel of target cell lines: Huh-7 human hepatocellular carcinoma (Nakabayashi et al., 1982), PLC/PRF/5 human hepatoma (CRL-8024), Hep3B human hepatocellular carcinoma (ATCC HB-8064), HepG2 human hepatocellular carcinoma (HB-8065), A431 human epidermoid carcinoma (CRL-1555), Caco-2 human colon adenocarcinoma (HTB-37), HCT 116 human colorectal carcinoma (CCL-247), HOS human osteosarcoma (CRL-1543), HT-1080 human fibrosarcoma (CCL-121), HT-29 human colorectal adenocarcinoma (HTB-38), LoVo human colorectal adenocarcinoma (CCL-229), MCF-7 human breast adenocarcinoma (HTB-22), 293T, TE671 human rhabdomyosarcoma (ATCC CRL-8805), U118 human glioblastoma (HTB-15), Jurkat human T cell leukemia (TIB-152), CEM human lymphoblastic leukemia (CCL-119), Molt-4 human lymphoblastic leukemia (CRL-1582), Raji Burkitt's lymphoma (CCL-86), CMMT Rhesus monkey mammalian carcinoma (CRL-6299), COS-7 African green monkey fibroblasts kidney (CRL-1651), VERO African green monkey kidney (CCL-81), PG-4 feline astrocyte (CRL-2032), BHK-21 golden hamster kidney (CCL-10), CHO Chinese hamster ovary (ATCC CCL-61), BRL 3A rat hepatocytes (CRL-1442), NIH3T3 mouse fibroblasts (CRL-1658) and QT6 quail fibrosarcoma (CRL-1708). Target cells were grown as recommended by the ATCC (American Type Culture Collection, Rockville, Md., USA).

Target cells (seeded the day before 8×10⁴ cells/well in 12-well plates) were incubated for 3 hrs with dilutions of supernatants from producer cells containing the HCVpp, then washed and cultured until expression of the GFP marker gene harboured by the virions was assessed by FACS analysis 72 hrs later (Sandrin et al., 2002). Since the HCVpp were generated with replication-defective viral components, this procedure allowed evaluation of the specific infectivity of the pseudo-particles after a one-round infection process.

Infectious titres of up to 1.1×10⁵ TU/ml were detected for the HCVpp on Huh-7 human hepato-carcinoma cells (FIG. 1). Upon concentration of the producer cell supernatants by ultra-centrifugation (Sandrin et al., 2002), infectious titres of 2×10⁶ TU/ml, on average, could be readily obtained. The other target cell types used in the infection assays displayed weaker (PLC/PRF/5, Hep3B, HepG2, Caco-2, HT1080, HT-29, LoVo, MCF-7, U118, 293T, Vero) or undetectable (A431, HCT 116, HOS, TE671, Jurkat, Molt-4, CEM, Raji, CMMT, Cos-7, BHK-21, CHO, PG-4, BRL 3A, NIH3T3, QT6) levels of infection with the HCVpp. The infectivity of control pseudo-particles generated with VSV-G ranged from 7×10⁶ to 2×10⁷ TU/ml depending on the target cell type indicating that all these cells were readily infected with the control pseudo-particles. This suggested that all the molecules necessary for HCV entry were specifically expressed in the former cell types only.

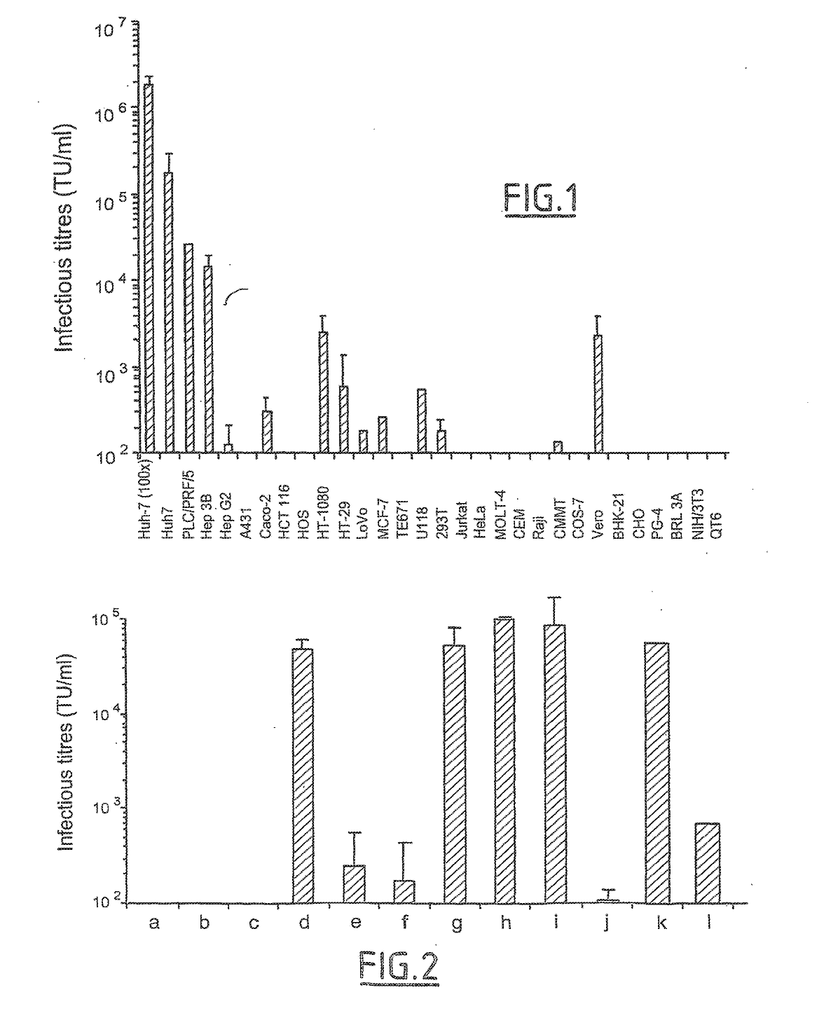

Infectious HCVpp could be generated at comparable efficiencies with E1E2 glycoproteins derived from HCV genotypes 1a and 1 b (lanes h and i; FIG. 2) and/or with retroviral core proteins derived from either HIV-1 or MLV (lanes d and h; FIG. 2).

Incubation of HCVpp and target cells with AZT, a reverse-transcriptase inhibitor that prevents conversion of the retroviral RNA genome of the HCVpp as integration-competent proviral DNA, inhibited transduction (lane j; FIG. 2).

Moreover, long-term expression of GFP could be demonstrated after serial passaging of the infected cells for more than one month.

These results indicated that infection of the target cells led to integration in host cell DNA and stable expression of the GFP marker gene transduced by the HCVpp. Additionally, no infection could be obtained with viral particles lacking both E1 and E2 glycoproteins, or lacking core proteins, or, alternatively, when using the MLV-G2A assembly-defective core proteins (lanes a-c; FIG. 2).

The inventors have further demonstrated that hepatic cell infection could be achieved with HCVpp harbouring E1 and E2 glycoproteins of genotype 2a, 2b, 3a, 3b, 4a and 4b.

Altogether, these results support that HCVpp harbouring the E1 and E2 glycoproteins of any genotype and retroviral core proteins are infectious, leading to retroviral-mediated integration of their vector genome.

Finally, despite reduced levels of E1 incorporation, HCVpp generated with E1 and E2 glycoproteins expressed in trans, from distinct vectors, were nearly as infectious as those generated with the E1E2 polyprotein construct (lanes g and h; FIG. 2). However, HCVpp assembled with either E1 or E2 glycoproteins only were 500-fold less infectious (lanes e-f; FIG. 2), demonstrating that both glycoproteins need to be co-incorporated on the HCVpp to allow efficient virus entry and infection.

### Example 3

### HCVpp Primary Hepatocyte Infectivity

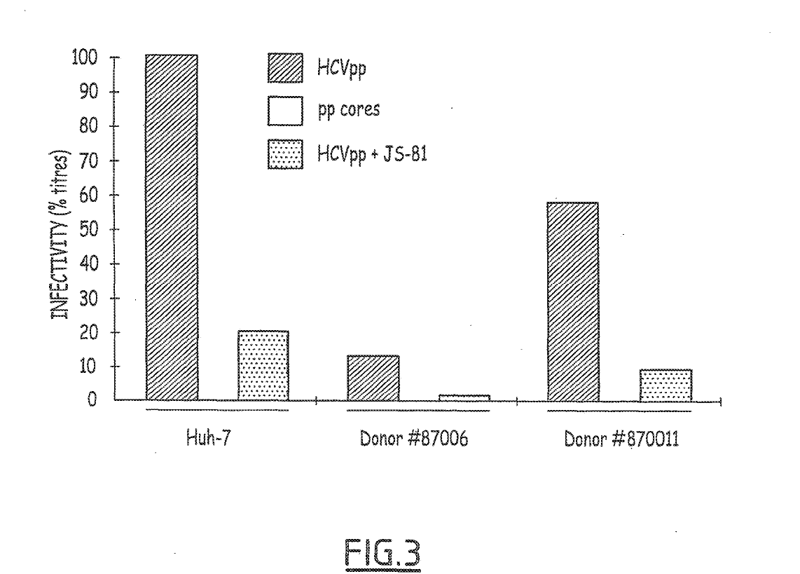

Hepatocytes represent the principal site of HCV replication in vivo, yet ex vivo studies have suggested that HCV may also infect lymphoid cells (Lindenbach and Rice, 2001). To address whether either cell types could be infected, human adult hepatocytes (FIG. 3) and peripheral blood mononuclear cells (PBMCs) have been transduced. Pseudo-particles were generated with core proteins derived from HIV-1 rather than from MLV, which do not permit transduction of non-proliferating target cells (Nègre et al., 2000).

Cryopreserved primary hepatocytes, isolated from human adult biopsy samples checked for absence of HBV, HCV and HIV, were purchased from Biopredic International (Rennes, France) and cultured on collagen I coated plates according to recommendations of the supplier. Transduction of hepatocytes was determined by counting GFP-positive cells under UV microscope. Specificity of infection was demonstrated by absence of transduction when target cells were infected with pseudo-particles lacking glycoproteins (pp cores) or by the reduced levels of transduction when target cells were pre-incubated with JS-81 anti-CD81 antibodies (30 μg/ml) before infection. Infection of human PBMCs, isolated from healthy donors, was conducted as described previously (Sandrin at al., 2002).

Relative to infection of Huh-7 cells, the HCVpp could readily infect the primary hepatocytes derived from different donors (FIG. 3), yet transduction efficiencies varied with quality and cell culture viability of the individual biopsies. In contrast, poor or undetectable infection of PBMCs was found with the HCVpp although control pseudo-particles generated with VSV-G could readily infect these primary cells.

Altogether these data indicate that HCVpp closely mimic the tropism and early events of infection by wild-type HCV and preferentially infect hepatocytes and hepato-carcinoma cells.

### Example 4

### HCVpp is a Valid Model of Early Steps of HCV Infection

Infectivity of HCVpp is Mediated by E1 and E2 Glycoproteins

It was further determined whether infectivity of the HCVpp is specifically mediated by E1 and E2 and their interaction with cell surface receptors. A panel of monoclonal antibodies previously shown to specifically react against HCV-1a glycoproteins (Dubuisson et al., 1994; Flint et al., 1999; Patel et al., 2000) was used.

HCVpp generated with HCV-1a E1E2 and with MLV core proteins were pre-incubated before infection of Huh-7 cells with saturating concentrations (20 μg/ml) of monoclonal antibodies against E1 (A4) or E2 (H31, 1-133, H35, H44, H48, H53, H54, H60 and H61) glycoproteins of genotype 1a, or with pooled antibodies (Hmix). Control experiments were performed using no antibodies or using pseudo-particles generated with VSV-G (VSV-Gpp).

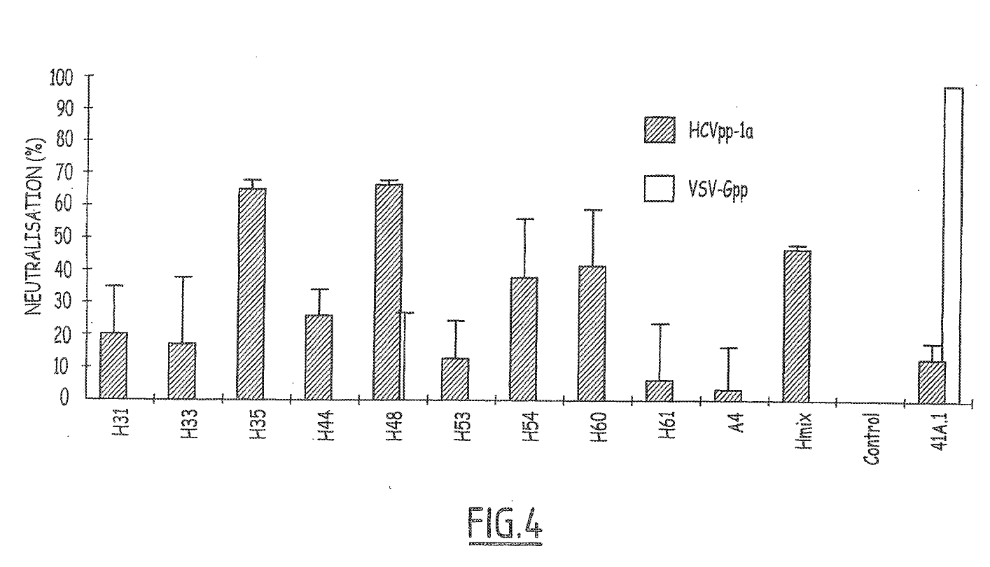

Some of these monoclonal antibodies, like H35 and H48, for example, could neutralise the HCVpp of genotype 1a and reduce their infectivity by up to 70% (FIG. 4). That incomplete neutralisation might be due to the fact that these monoclonal antibodies, which were developed and selected for binding to intra-cellular E1E2 complexes, have been shown to be sensitive to post-translational modifications of E2 (Flint M. et al., 2000). Indeed, compared to its intra-cellular counterpart, E2 associated to HCVpp was found to have undergone sugar modifications, most likely as a result of its export through the cell secretory pathway.

In contrast, none of these genotype 1a-specific antibodies could neutralise the HCVpp of genotype 1b or the control pseudo-particles generated with VSV-G (FIG. 4). Neutralisation of the infectivity of these control pseudo-particles was achieved by using the VSV-G neutralising 41A.1 monoclonal antibody.

These data therefore demonstrate that infectivity of the pseudo-particles is specifically due to the incorporation of E1 and E2 glycoproteins, indicating that these HCVpp represent a valid model to investigate the early steps of HCV infection, i.e. receptor binding, membrane fusion and envelope uncoating.

HCV Infection is Neutralised by Serum from HCV-Infected Patients

The capacity of sera derived from chronically HCV-infected patients to neutralise infectivity of HCVpp was further assessed.

HCVpp of genotypes 1a or 1 b were pre-incubated for 30 min at room temperature with sera from chronically HCV-infected patients diluted 1/50 before infection of Huh-7 target cells. Control experiments were performed using pseudo-particles generated with RD114 glycoproteins (RD114pp), rather than with VSV-G that exhibits sensitivity to human complement (Sandrin et al., 2002). Efficient neutralisation of the control pseudo-particles was demonstrated using a hyper-immune goat serum raised against the RD114 SU glycoprotein (ViroMed Biosafety Laboratories, USA).

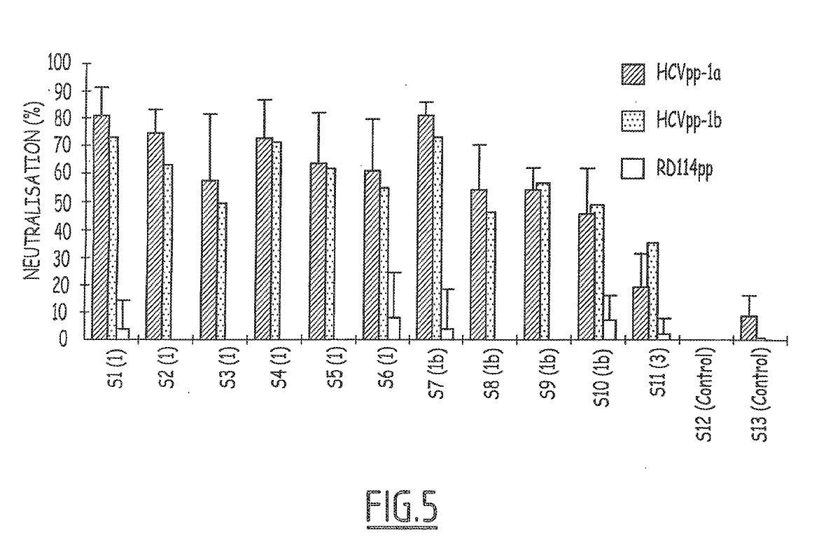

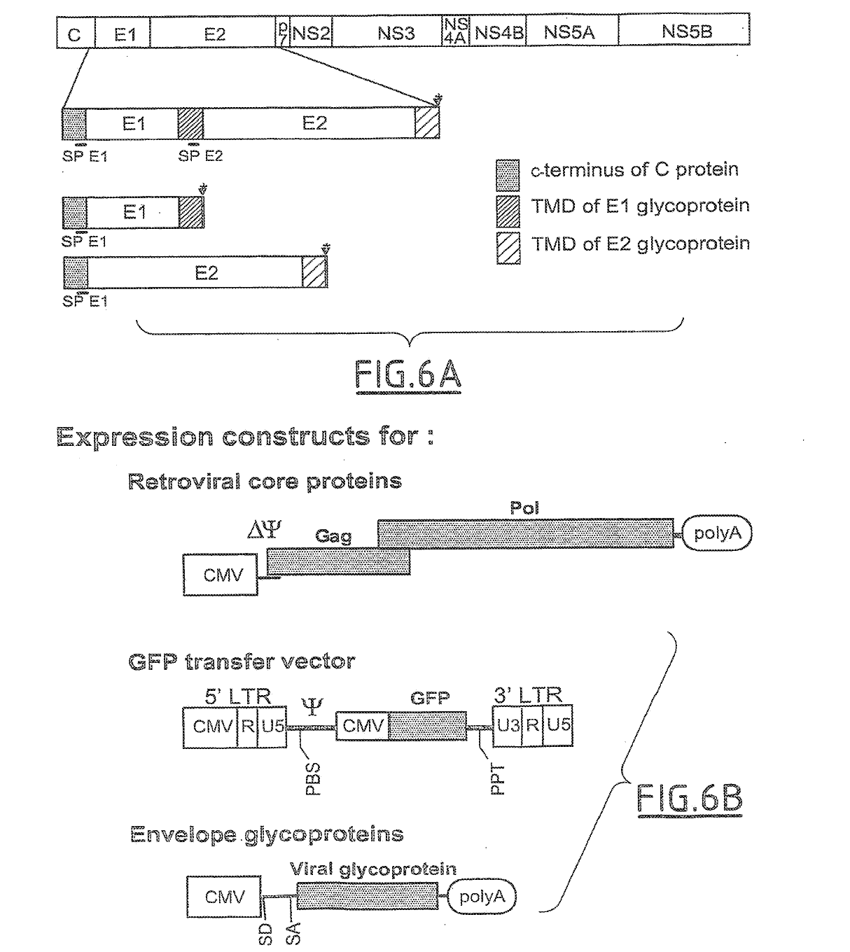

No or only weak neutralisation of control pseudo-particles could be detected using the sera of the HCV-infected patients. In contrast, most if not all of these sera could neutralise the infectivity of the HCVpp, in contrast to sera derived from healthy donors. Variable levels of neutralisation were detected depending on the donor and ranged from 20% to up to 90% of inhibition for HCVpp of both genotypes 1a and 1b. Sera derived from patients infected with HCV of the 1b genotype could neutralise the HCVpp generated with E1E2 of genotype 1a or 1 b with similar efficiencies (FIG. 5).

### Example 5

### Cell Entry Receptor Usage by HCV Pseudo-Particles

Both the LDL receptor (LDLr) and CD81, a member of the tetraspanin family of receptors, have been proposed as putative HCV receptors (Pileri et al., 1998; Agnello et al., 1999). However, recent studies have questioned the role of these molecules as cell entry receptors despite their unequivocal capacity to bind HCV particles and/or glycoproteins (2023). Thus, the contribution of either cell surface molecules in the early stages of HCV cell entry was investigated by performing receptor-competition assays.

More recently, SR-B1 the human scavenger receptor class B type I expressed in steroidogenic tissues has been put forward as another putative HCV receptor (Scarselli, Ansuini et al. 2002). Thus, the contribution of SR-BI in the early stages of HCV cell entry was investigated by performing infection assays in non-permissive target cells engineered to express this HCV receptor candidate.

LDLr

LDLr binds apoliprotein B within LDL and apoliproteins B and E (ApoB and ApoE) within VLDL complexes; both complexes have been found associated with HCV particles in plasma of infected patients (Andre et al., 2002).

LDL receptor competition assays were performed using purified LDLs (10 μg/ml, 100 μg/ml; Sigma-Aldrich, France) or with the monoclonal antibodies 5E11 (10 μg/ml, 50 μg/ml), 4G3 (anti-ApoB; 10 μg/ml, 50 μg/ml) or 1B7 (anti-ApoE; 1 μg/ml, 10 μg/ml, 50 μg/ml, 100 μg/ml) (Ottawa Heart Institute Research corporation, Ottawa, Ontario, Canada), or mixed 5E11, 4G3 and 1B7 antibodies (with individual concentrations 10 μg/ml, 50 μg/ml), pre-incubated with the pseudo-particles prior to infection.

Purified LDLs were not found to out-compete infection of HCVpp on Huh-7 LDLr-positive cells, even when concentrations higher than 100 μg/ml were used. When monoclonal antibodies targeted to the receptor binding sites of ApoB and ApoE were pre-incubated with the HCVpp, only the anti-ApoE antibodies could inhibit, albeit weakly, the infectivity of the HCVpp. This is consistent with our observation that HCVpp could not or hardly infect LDLr-positive cell lines such as HepG2, Jurkat, CEM, Molt-4, Raji and TE671 (FIG. 1). Thus, although these results do not exclude a role of LDLr in HCV entry, possibly via association of LDLs with HCV particles (Andre et al., 2002), they suggest that LDLr is not the major receptor of HCVpp entry.

CD81

Recombinant GST-fusion polypeptides encompassing the large extra-cellular loop (LEL) of human CD81 which has been shown to bind HCV E2 (Flint et al., 1999, (Piled et al., 1998) were then used to investigate the role of hCD81 in HCVpp cell entry.

hCD81-LEL GST-fusion polypeptides (4 μg/ml, 8 μg/ml, 16 μg/ml) were pre-incubated with the pseudo-particles before infection or JS-81 anti-CD81 antibodies (4 μg/ml, 10 μg/ml, 30 μg/ml; Pharmingen, France) were pre-incubated with the target cells prior to infection. Percentages of inhibition of the infectious titres obtained on Huh-7 cells relative to titres obtained in the absence of inhibitors were calculated. Control experiments were performed using pseudo-particles generated with VSV-G (VSV-Gpp). Comparative experiments were further conducted with NIH3T3, NIH3T3-hCD81 and Huh-7 cells infected with HCVpp and with control pseudo-particles generated with VSV-G.

The GST-fusion hCD81-LEL polypeptides pre-incubated with the HCVpp could specifically precipitate E1E2 complexes, confirming their capacity to bind the E2 glycoprotein (Flint et al., 1999; Pileri et al., 1998), and were found to neutralise the infectivity of the HCVpp on Huh-7 cells, yet with a relatively poor efficiency. Inhibition ranged from 38% to 54% inhibition of infectivity and appeared to be dose-dependent. Consistently, pre-incubation of Huh-7 cells with JS-81 monoclonal antibody, that blocks binding of recombinant E2 to hCD81 (Flint et al., 1999), was found to inhibit infectivity of HCVpp in both Huh-7 target cells and human primary hepatocytes. At high antibody concentrations (30 μg/ml), over 90% inhibition of infection could be obtained for HCVpp based on genotypes 1a as well as 1 b. However, several target cells non-permissive to infection by HCVpp, like TE671, Jurkat, CEM, Molt-4 and Raji cells (FIG. 1) were found to express high densities of hCD81, indicating the lack of correlation between CD81 expression and infectivity.

Moreover, expression of hCD81 in non-permissive NIH3T3 mouse fibroblasts did not allow infection with the HCVpp (FIG. 4C).

Altogether these results demonstrate that although hCD81 binds HCV E2 and might help cell surface attachment of HCV, it is not sufficient by itself to allow infection with HCVpp.

SR-B1

A cDNA encoding SR-BI, also known as CLA-1 (CD36 and LIMPII Analogous-1) (Calvo and Vega, 1993; Webb et al., 1998) was introduced in an MLV retroviral vector and vector particles were generated and pseudotyped with the VSV-G glycoprotein. They were used to transduce CHO and 3T3 cells, that are non permissive to HCVpp infection, as shown in FIG. 1. Expression of SR-BI in these transduced cells was verified by FACS analysis using antibodies, directed against the SR-BI ectodomain. The SR-BI-transduced cells as well as the parental cells were used as target cells for infection assays using the HCVpp. Huh7 cells were used as control permissive target cells.

In contrast to the Huh-7 cells, neither the SR-BI-transduced cells nor the parental CHO or 3T3 cells could be infected with HCVpp. These data indicates that although SR-B1 binds E2 (Scarselli et al., 2002), it is not sufficient to allow entry of HCVpp in cells.

Therefore, as for hLDLR and hCD81, expression of SR-B1 expression alone is not enough to render cell permissive to infection.

To address the possibility that HCV target cells need to co-express all molecules to allow HCVpp infection, CHO and 293 target cells, that respectively have no or low SR-B1 expression and that are both non permissive to infection with the HCVpp (Bartosch et al., 2003 a and b), were co-infected with two VSV-G-pseudotyped retroviral vectors carrying human CD81 and SR-BI, respectively. Co-expression of both cell suface molecules on either cell type was verified two days after transduction, by FACS analysis.

The CD81 and SR-BI co-expressing CHO or 293 cells were then used as target cells for infection assays using the HCVpp. Similar to parental CHO cells and to the same cells individually transduced by either CD81- or SR-BI-expression retroviral vectors, this resulted in absence of infectivity. In contrast to parental 293 cells as well as to CHO-CD81/SR-B1 cells, SR-B1-transfected 293 cells were found permissive to infection, indicating that co-expresssion of CD81 and SR-B1 is required for infection in human cells.

Since HepG2 human hepato-carcinoma cells are non-permissive to infection by the HCVpp, the inventors further thought that their non-permissiveness could be due to lack of co-expression of both CD81 and SR-BI. Indeed, HepG2 cells express SR-BI but lack CD81 expression.

Thus the HepG2 cells were transduced with a CD81-carrying MLV retroviral vector pseudotyped with VSV-G. CD81-expression was verified by FACS analysis two days after transduction and the CD81-transduced HepG2 cells were then used as target for infection assays using the HCVpp.

Compared to the parental HepG2 cells which were poorly infected with the HCVpp, ectopic expression of CD81 in these cells resulted in high infectivity of the HCVpp, similar to that observed in Huh-7 cells.

### Example 6

### Deletion of Hypervariable Region I

HCV infection is not cleared by over 80% of patients and chronicity is associated with various forms of liver disease. Chronic infection is thought to be caused by the high mutation rate of HCV, which helps the virus to escape the host immune response. One of several mutation hotspots within the HCV genome has been localised to the N-terminus of the E2 glycoprotein. This so-called hypervariable region I is a major B cell epitope and a target of neutralising antibodies in vivo. Indeed HCVpp can be very efficiently neutralised by mouse monoclonal antibodies or by rabbit hyper-immune sera raised against HVR1 (Bartosch et al., 2003b). However, such antibodies are highly specific for the homologous sequence and do not neutralize slightly divergent HVR1 variants of the virus as well as ΔHVR1 HCVpp (Bartosch et al., 2003b). Thus, because of its variability, HVR1 is thought to play an important role in allowing HCV to escape the host's immune response. The antigenic variation of the HVR1 may also inhibit the development of a protective immune response against re-infection with heterologous HCV strains/genotypes. Finally does the antigenic dominance and extreme variability of the HVR1 hinder the development of vaccines against HCV. With the long-term aim to develop effective therapeutics which target more conserved parts of the HCV glycoproteins the inventors investigated whether the HVR1 is dispensable for infection.

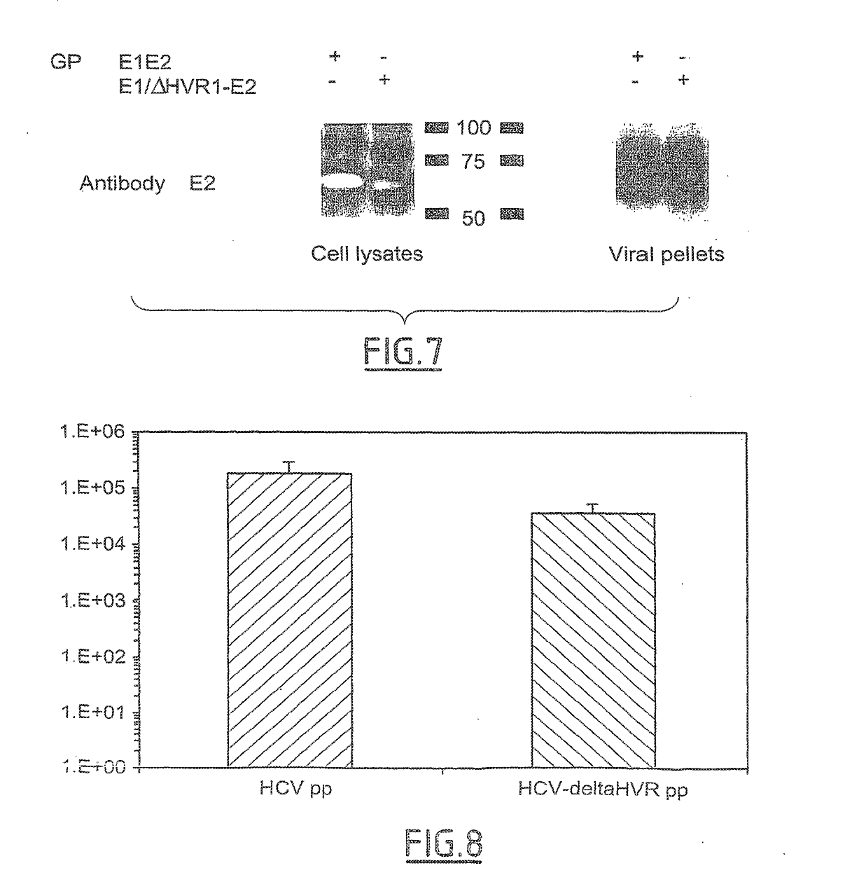

For that purpose expression constructs encoding the polyprotein for wildtype E1E2 or a mutant version in which the HVR1=the first 27 residues of E2 had been deleted from the polyprotein were generated. HCV pseudo-particles (HCVpp) were produced with either wildtype or ΔHVR1 ΔCE1E2 polyprotein. Analysis of immunoblots of transfected cells showed that both wildtype and mutant E2 proteins were expressed to similar levels. Viral particles were harvested from the supernatant of transfected cells and purified by ultra-centrifugation through high-density sucrose cushions. ΔHVR1 and wild-type E2 were incorporated into particles to a similar level (FIG. 7). The infectivity of the wildtype and mutant HCVpp were tested as before on Huh-7 target cells. Infectious titres of up to 2.6×10⁵ TU/ml were detected for wildtype HCVpp (FIG. 8). ΔHVR1 HCVpp were about 5 to 10 fold less infectious, still a maximal titer of 5.1×10⁴ TU/ml was observed suggesting that the HVR1 aids the infection process, via interaction to SR-B1 receptor (Scarselli et al., 2002: Bartosch et al., 2003b).

ΔHVR1 HCVpp will be useful not only to induce antibodies against more conserved neutralising epitopes but also as a tool to detect neutralising epitopes that are target of the humoral response induced by HCV infection. The humoral response in sera from some HCV-infected patients and from experimentally inoculated chimpanzees has been characterised (Bartosch et al., 2003c). Neutralizing antibodies from chronic infections were relatively high-titered against the homologous and a heterologous genotype 1a pp and usually, but not always, cross-neutralized a pseudotyped virus of a different subgenotype. To determine if the pedigreed sera tested herein reacted with epitopes outside of the HVR1 region of the HCV envelope proteins, the panel of sera was tested at a dilution of 1:50 against the ΔHVR1 HCVpp. All of the sera that neutralized parental HCVpp also neutralized ΔHVR1 HCVpp. Thus, neutralizing antibodies from patients and chimpanzees persistently infected with HCV reacted with one or more additional epitopes elsewhere in the HCV envelope glycoproteins. The observation that chronically infected chimpanzees and humans do make neutralizing anti-HCV antibody and that this antibody is broadly cross-reactive, is consistent with the observation that immune globulin prepared from such chronically infected patients can protect against HCV infection. This has important implications for rethinking the feasibility of developing antibody-based passive and active immunophophylaxis strategies against HCV as well as for reassessing the relative importance of humoral and cellular immunity in controlling chronic HCV infections.

### Example 7

### HCVpp and Cell-Cell Fusion Assays

Cell-cell fusion assays were designed by co-culturing E1E2-transfected 293T (donor) cells with target (effector) cells. The effector cells were hepato-carcinoma cells or, as control, 293T cells.

Transformed cells, transfected with the constructs expressing the envelope glycoproteins, are detached, counted and re-seeded at the same concentration (3×10⁵ cells/well) in six-well plates. Fresh target host cells (1×10⁶ cells per well) are then added onto the transfected cells and are cocultivated for 24 hours.

The coculture is submitted to a acidic pH drop by replacing the culture medium with a pH5-buffered DMEM for 5 min and incubating for 5 min Acidic medium was then replaced with a normal (neutral pH medium) and cultures were grown for 12 hours until cell-cell fusion was evaluated by scoring the syncytia.

Cultures are then stained by adding the May-Grunwald and Giemsa solutions (MERCK) according to the manufacturer recommendations. Cells containing two or more nuclei can be defined as syncytia. A fusion index is then defined as the percentage of (N−S)/T where N is the number of nuclei in the syncytia, S is the number of syncytia and T is the total number of nuclei counted.

The results of syncytia scoring are displayed in Table 1.

TABLE 1
Results of syncytia assays with Huh-7 cells

| Transfected | Syncytia at | Syncytia |
|---|---|---|
| glycoprotein | neutral pH¹ | after pH-5 shock¹ |
| None | − | − |
| MLV-A | − | − |
| MLV-A-Rless | ++ | ++ |
| FPV-HA | − | ++ |
| VSV-G | + | +++ |
| HCV E1 | − | − |
| HCV E2 | − | − |
| HCV E1E2 | − | ++² |

¹(−), fusion index of less than 1%, (+), fusion index between 1% and 5%; (++), fusion index between 5% and 20%; (+++), fusion index higher than 20%.
²no syncytia were detected when HCV E1E2-transfected cells were co-cultivated with 293 cells after a pH-5 shock

Expression of control amphotropic murine leukaemia virus (MLV-A) pH-independent glycoproteins did not result in syncytia in these experimental conditions. However, extensive syncytia formation was detected when using a mutant form of MLV-A glycoprotein with shortened cytoplasmic tail (MLV-A-Rless).

Expression of pH-dependent fowl plague virus hemagglutinin (FPV-HA) resulted in syncytia formation only when the co-cultures were incubated for 5 min at pH-5.

Expression of the pH-dependent G glycoprotein of VSV (vesicular stomatitis virus) resulted in small syncytia formation at neutral pH and in extensive syncytia formation at acid pH.

Although expression of HCV E1 or HCV E2 alone did not results in syncytia under either experimental conditions, co-expression of both E1 and E2 in 293T cells co-cultivated with Huh-7 cells was sufficient to induce the formation of syncytia. This indicates that under such experimental conditions, HCV E1E2 can fuse cells expressing the appropriate HCV receptors.

Moreover these results indicate that unlike most retroviral glycoproteins (eg., MLV-A), but like influenza virus hemagglutinin (eg., FPV-HA) or VSV-G, fusogenicity of HCV E1E2 is triggered by acid pH. These findings are confirmed by the sensitivity of the HCV pseudo-particles to inhibitors of endosomal acidifications (Table 2).

TABLE 2
pH-sensitivity of infection by HCV pseudo-particles

Bafilomycin-A
concentration (nM)²

| Glycoprotein¹ | 0 | 50 | 100 | 200 |
|---|---|---|---|---|
| MLV-A | 100 | 91.2 | 70 | 45 |
| FPV-HA | 100 | 12.8 | 2.3 | 2.7 |
| VSV-G | 100 | 27 | 7.3 | 0.5 |
| HCV-E1E2 | 100 | 12.8 | 4.3 | 3 |

¹GFP-carrying pseudo-particles generated with the indicated glycoprotein were used to infect Huh-7 cells in the presence of the indicated concentration of Bafilomycin A. The inhibitor was removed after 3 hr and infectivity was determined by FACS analysis 3 days later.
²results of infection for the indicated concentration of Bafilomycin-A are expressed as percentage of the infectivity determined in the absence of inhibitor.

Thus, this novel cell-cell HCV fusion assay is highly valuable for the screening of molecules capable to inhibit HCV binding and cell entry by membrane fusion.

### Example 8

### Assembly and Increased Infectivity of HCV Pseudo-Particles in the Presence of HCV p7

The p7 protein may influence the conformational changes of the HCV envelope proteins that are perhaps important for cell binding and entry of the HCV virion. Accordingly, to gain more information on the p7 protein, expression constructs expressing a E1E2p7 polyprotein were generated.

The phCMV-ΔCE1E2p7 (phCMV 7a p7-1) expression vector expressing HCV 1a proteins delta core, E1, E2 and p7 was constructed using the Stu I, Bcl I digested fragment from plasmid pTM1p5E1E2p7 (Fournillier-Jacob et al, 1996; Cocquerel et al, 2000), comprising E1, E2 and p7 of HCV genotype 1a. This fragment was ligated into the previously described plasmid phCMV-7a after Bsu36 I digestion, blunting and subsequent digestion with Bcl I. The sequence of the phCMV-ΔCE1E2 vector is shown in SEQ ID No 14, whereas the aminoacid sequence of the ΔCE1E2 polyprotein is shown in SEQ ID No 15.

Infectivity of the HCV pseudo-particles generated with p7 (HCV-p7pp) was compared with that of HCVpp. Expression of p7 during assembly of the HCV pseudo-particles resulted in up to 10-fold increased titres. Similar increase of infectious titres mediated by p7 expression were obtained for HCVpp assembled with E1E2 glycoproteins derived from HCV genotypes 1a and 1 b.

### Example 9

### Deletion of the C-Terminal Amino Acid Residue of Mature E2 Glycoprotein

The fragment of the HCV polyprotein used to express the ΔCE1E2 polypeptide is usually numbered relative to the total HCV polyprotein. For the 1a strain H HCV genotype (accession number in GeneBank: AF009606), ΔC is therefore encompassed by amino-acids 132-191 (signal peptide: amino-acids 171-191), E1 by amino-acids 192-383, and E2 by amino-acids 384-746 (HVR1: amino-acids:384-410).

The inventors have co-expressed this full length polypeptide, from amino-acids 132 to 746 (with a short sequence, MNS, fused to the amino-terminus of ΔC and that is encoded by the translation initiation site of the ΔCE1E2 polypeptide gene) with the other components required to assemble infectious HCV pseudo-particles. HCV pseudo-particles can also be assembled with a shorter version of the ΔCE1E2 polypeptides, encompassing amino-acids 132-745 (and the MNS amino-terminal peptide).

Both types of HCV pseudo-particles, ie. those carrying the full length E1E2 glycoproteins (HCVpp(746)) and those carrying E1E2 glycoproteins deleted of the last amino-acid at position 746, an alanine, (HCVpp(745)) incorporate normal level of glycoproteins. However, the HCVpp(745) pseudo-particles are consistently 10-50 times more infectious than the HCVpp(746) pseudo-particles. This improvement in infectivity is therefore important in infection assays as well as in screening of neutralising compounds and antibodies.

### Example 10

### Mice Immunization with HCVpp Elicit Neutralizing Antibodies

Balb/c mice, aged 4-5 weeks, in groups of 4 animals, received intraperitoneal injections of purified HCVpp harbouring E1E2 glycoproteins of genotype 1a, 1b, 2a or HCVpp ΔHVR1 ΔCE1E2, as described in example 6, or as control, with pseudo-particles devoid of viral surface glycoproteins or carrying a non-relevant glycoprotein, from RD114 feline endogenous virus (Bartosch et al., 2003a). About 1e6 purified HCVpp, corresponding roughly to 1e9 physical particles, were used per mice. Each mouse was boosted two times with identical inoculae. The sera of the animals were harvested and their neutralising activity was compared to that of control sera, harvested from the same animals before immunisation, in infection assays using the HCVpp.

Neither these control sera nor the sera from animals immunised with the control pseudo-particles could neutralise the infectivity of the different types of HCV pseudo-particles. In contrast, the sera of mice immunised with the HCV pseudo-particles could efficiently neutralise the infectivity of both the HCV pseudo-particle type matching that used for immunisation and the other types of HCV pseudo-particles. This resulted in over 90% inhibition of infectivity when sera of mice immunised with the HCVpp were diluted to 1/80 before incubation with the HCVpp.

These data indicated that HCV pseudo-particles are useful to elicit the formation of neutralising antibodies as well as cross-neutralising antibodies. The ability to induce neutralizing anti-HCV antibodies should permit an assessment of the prospects for successful antibody-mediated passive and active immunoprophylaxis against hepatitis C.

In conclusion, the invention provides a tool that allows precise investigation of viral assembly of E1E2 glycoproteins (processing, maturation) and their role in cell entry of HCV. No structural modifications of the E1E2 glycoproteins were required for their correct assembly on retroviral and lentiviral cores and to generate high titer infectious HCVpp with functional E1E2 glycoproteins. Deletion of the C-terminal residue of HCV E2 protein was further found to greatly enhance infectivity of the generated HCV pseudo-particles. The insertion of a marker gene into the HCVpp allowed precise and rapid determination of the infectivity of these pseudo-particles by flow-cytometry.

The development of these functional, infectious HCV pseudo-particles make it now possible to investigate early events of HCV infection, such as the identification of novel HCV receptor(s) or co-receptor(s), to carry out diagnostic assays for the detection of neutralising antibodies in seroconverted patients, and to develop efficient inhibitors to HCV infection.

### REFERENCES

Agnello V. et al., Proc Natl Acad Sci USA 96, 12766-71. (1999).
Andre P. et al., J Virol 76, 6919-28. (2002).
Ausubel F. M. et al. (eds.), Current Protocols in Molecular Biology, John Wiley & Sons, Inc. (1994).
Bartosch, B., Dubuisson J. & Cosset, F.-L. 2003a. J. Exp. Med. 197, 633-642.
Bartosch et al, J. Biol. Chem. 2003b Aug. 11 [Epub ahead of print].
Bartosch et al., 2003c. Submitted.
Baumert T. F., Ito S., Wong D. T., Liang T. J., J Virol 72, 3827-36. (1998).
Blight K. J., Kolykhalov A. A., Rice C. M., Science 290, 1972-4. (2000).
Buonocore L., Blight K. J., Rice C. M., Rose J. K., J Virol 76, 6865-72. (2002).
Calvo and Vega (1993) J. Biol. Chem. 268:18929-18935
Castet V, Moradpour D. Hepatology. 2003 September; 38(3):771-4.
Cocquerel et al., J Virol, 74/8, 3623-3633 (2000)
Dieterich (2002) J Infect Dis 185, S128-37.
Dubuisson J. et al., J Virol 68, 6147-60. (1994).
Feigner et al., Science 337, 387-388. (1989).
Flint M. et al., J Virol 73, 6235-44. (1999).
Flint et al., J Virol 74, 702-709 (2000).
Fournillier-Jacob et al., J Gen Virol, 77, 1055-1064 (1996)
Guguen-Guillouzo and Guillouzo. Methods for preparation of adult and fetal hepatocytes. p 1-12. In A. Guillouzo and C. Guguen-Guillouzo (ed.), Isolated and cultured hepatocytes. Les Editions INSERM Paris. John Libbey and Co, Ltd., London, United Kingdom (1986).
Hsu, M., Zhang, J., Flint, M., Logvinoff, C., Cheng-Mayer, C., Rice, C. M. & McKeating, J. A. (2003) Proc. Natl. Acad. Sci. USA 100, 7271-7276.
Lavanchy D. et al., J. Viral Hepat 6, 35-47 (1999).
Lindenbach B. D., Rice C. M., Flaviviridae: The Viruses and Their Replication. Knipe D. M., Howley P. M., Eds., Fields Virology, 4th ed. (Lippincott Williams & Wilkins, Philadelphia, Pa., 2001).
Lohmann V. et al., Science 285, 110-3. (1999).
Matsuura Y. et al., Virology 286, 263-75. (2001).
Nakabayashi H. et al., Cancer Res 42, 3858-63. (1982).
Nègre D. et al., Gene Ther 7, 1613-1623 (2000).
Op De Beeck A. et al., J Biol Chem 275, 31428-37. (2000).
Op De Beeck A., L. Cocquerel, J. Dubuisson, J Gen Virol 82, 2589-95. (2001).
Owsianka A. et al., J Gen Virol 82, 1877-83. (2001).
Paetzel et al. (2002)). Chem Rev. December; 102(12):4549-80
Patel A. H. et al., J Gen Virol 81, 2873-83. (2000).
Pietschmann T. et al., J Virol 76, 4008-21. (2002).
Pileri P. et al., Science 282, 938-41. (1998).
Robertson et al. (1998) Archives of Virology 143, 2493-2503
Rose N. F. et al., Cell 106, 539-49. (2001).
Sambrook, J., Fritsch, E. F., and Maniatis, T. Molecular Cloning: A Laboratory Manual, 2nd Ed., Cold Spring Harbor Laboratory, Cold Spring Harbor, N.Y. (1989).
Sandrin V. et al., Blood 100, 823-832 (2002).
Scarselli, E. et al., EMBO J. 21: 5017-25 (2002).
Swanstrom R., Wills J. W., in Retroviruses J. M. Coffin, S. H. Hughes, H. E. Varmus, Eds. (Cold Spring Harbor Laboratory Press, New York, USA, 1997) pp. 263-334.
von Heijne G. (1990) J Membr Biol. 115(3):195-201
Wellnitz S. et al., J Virol 76, 1181-93. (2002).
Webb et al., J. Biol. Chem., 273:15241-15248. (1998)
Wu et al., J. Biol. Chem. 263:14621-14624. (1988)
Wu et al., J. Biol. Chem. 267:963-967. (1992)
Yagnik et al. (2000) Proteins 40, 355-366

1. A method for producing infectious hepacivirus-like particles ex vivo comprising the steps of:
providing a first nucleic acid sequence comprising a packaging competent retroviral-derived genome;
providing a second nucleic acid sequence comprising a cDNA encoding core proteins from said retrovirus;
providing a third nucleic acid sequence comprising a cDNA encoding a polyprotein comprising successively a signal peptide from a type I membrane protein, and a hepacivirus E1 protein and/or a hepacivirus E2 protein;
transfecting host cells with said nucleic acid sequences and maintaining the transfected cells in culture for sufficient time to allow expression of the cDNAs to produce structural proteins from hepacivirus and retrovirus; and allowing the structural proteins to form virus-like particles.

2. The method according to claim 1, wherein said third nucleic acid sequence comprises a cDNA encoding a polyprotein comprising successively a hepacivirus core protein, and a hepacivirus E1 protein and/or a hepacivirus E2 protein.

3. The method according to claim 1 or 2, wherein said packaging competent retroviral-derived genome and core proteins are from a retrovirus selected from the group consisting of MLV, ALV, RSV, MPMV, HIV-1, HIV-2, SIV, EIAV, CAEV, or HFV.

4. The method according to claim 2 or 3, wherein said polyprotein comprises a hepacivirus core protein and a hepacivirus E1 protein.

5. The method according to any of claims 2 to 4, wherein said polyprotein comprises a hepacivirus core protein and a hepacivirus E2 protein.

6. The method according to any of claims 1 to 5, wherein said third nucleic acid sequence comprises a cDNA encoding a polyprotein that further comprises a hepacivirus p7 protein.

7. The method according to any of claims 1 to 6, wherein said polyprotein comprises native hepacivirus E1 and/or E2 protein, and optionally native hepacivirus p7 protein.

8. The method according to any of claims 2 to 7, wherein said polyprotein comprises a native hepacivirus core protein, a native hepacivirus E1 protein and native hepacivirus E2 protein, and optionally a native p7 protein.

9. The method according to any of claims 2 to 8, wherein core protein, E1 protein and E2 protein, and optionally p7 protein, are derived from a same hepacivirus.

10. The method according to claim 9, wherein said hepacivirus is a hepatitis C virus (HCV).

11. The method according to claim 10, wherein said HCV core protein comprises the last 21 amino acids of the carboxy-terminus of HCV core.

12. The method according to claim 9 or 10, wherein said E2 protein is a mutated E2 protein selected from the group consisting of a E2 protein deleted from its C-terminal amino acid residue, and a native E2 protein wherein the hypervariable region 1 (HRV1) has been deleted.

13. The method according to any of preceding claims, wherein said nucleic acid sequence comprising a packaging competent retroviral-derived genome further comprises a transgene.

14. An infectious hepacivirus-like particle susceptible to be obtained by a method according to any of preceding claims, comprising the core proteins from a retrovirus, and a E1 hepacivirus glycoprotein and/or a E2 hepacivirus glycoprotein.

15. The infectious particle according to claim 14, comprising E1 and E2 hepacivirus glycoproteins.

16. The infectious particle according to claim 14, comprising E1 hepacivirus glycoprotein.

17. The infectious particle according to claim 14, comprising E2 hepacivirus glycoprotein.

18. The infectious particle according to any of claims 14 to 17, further comprising a hepacivirus p7 protein.

19. The infectious particle according to any of claims 14 to 18, comprising native E1 and/or E2 hepacivirus glycoprotein, and optionally native p7 protein.

20. The infectious particle according to any of claims 14 to 19, wherein core E1 and E2 protein, and optionally p7 proteins, are derived from a same hepacivirus.

21. The infectious particle according to claim 20, wherein said hepacivirus is HCV.

22. The infectious particle according to claim 21, wherein said E2 protein is a mutated E2 protein selected from the group consisting of a native E2 protein deleted from its C-terminal amino acid residue, and a native E2 protein wherein the hypervariable region 1 (HRV1) has been deleted.

23. The infectious particle according to any of claims 14 to 23, wherein said retrovirus is selected from the group consisting of MLV, ALV, RSV, MPMV, HIV-1, HIV-2, Sly, EIAV, CAEV, or HFV.

24. The infectious particle according to any of claims 14 to 23, wherein said nucleic acid sequence comprising a packaging competent retroviral-derived genome further comprises a transgene.

25. Use of three nucleic acid sequences for the preparation of a medicament useful as a vaccine against hepatitis, wherein the nucleic acid sequences are:
a first nucleic acid sequence comprising a packaging competent retroviral-derived genome;
a second nucleic acid sequence comprising a cDNA encoding core proteins from said retrovirus;
a third nucleic acid sequence comprising a cDNA encoding a polyprotein comprising successively a signal peptide from a type I membrane protein, and a hepacivirus E1 protein and/or a hepacivirus E2 protein;
and, when transferred into cells of a subject, the nucleic acids sequences allow the production of structural proteins from hepacivirus and retrovirus, wherein the structural proteins form virus-like particles that are immunogenic.

26. The use according to claim 25 wherein said third nucleic acid sequence comprises a cDNA encoding a polyprotein comprising successively a hepacivirus core protein, and a hepacivirus E1 protein and/or a hepacivirus E2 protein.

27. The use according to claim 25 or 26, wherein said third nucleic acid sequence comprises a cDNA encoding a polyprotein that further comprises a hepacivirus p7 protein.

28. The use according to any of claims 25 to 27, wherein said hepacivirus is HCV.

29. A method for ex vivo identification of a receptor for hepacivirus E1 and/or E2 glycoprotein comprising detection of the binding of an infectious particle according to any of claims 14 to 23, to a cell receptor.

30. A method for ex vivo identifying a cell receptor for hepacivirus comprising the step consisting of:
transfecting a cell which is not permissive for hepacivirus infection with a nucleic acid sequence encoding a protein likely to be a receptor for hepacivirus;
contacting said transformed cell with a hepacivirus-like particle according to any of claims 14 to 23;
determining whether said transformed cell has become permissive or not for hepacivirus infection; and
identifying as a cell receptor for hepacivirus said protein expressed by the transformed cell that has become permissive.

31. A method for ex vivo identifying a cell receptor for a hepacivirus comprising the step consisting of:
providing an expression cDNA library obtained from a cell permissive for hepacivirus infection;
transfecting cells that are not permissive for hepacivirus infection with said expression cDNA library;
contacting said transformed cells with hepacivirus-like particles according to any of claims 14 to 23;
identifying and isolating those transformed cells that have become permissive for hepacivirus infection;
isolating the expression vector transfected in cells that have become permissive; and
identifying as a receptor for hepacivirus the proteins encoded by the cDNA sequence of said isolated expression vectors.

32. A method of ex vivo screening or identification of molecules capable of interfering with hepacivirus entry in cells comprising comparison of the level of cell infection by an infectious particle according to any of claims 14 to 23 in the presence or the absence of a candidate molecule.

33. A method of in vitro diagnosis of a hepacivirus infection in a patient, comprising detecting immune complexes formed by interaction of anti-hepacivirus antibodies likely to be present in a biological sample of the patient with hepacivirus-like particle according to any of claims 14 to 23.

34. A method of in vitro diagnosis of a hepacivirus infection in a patient, comprising detecting an inhibitory effect of anti-hepacivirus antibodies likely to be present in a biological sample of the patient, on the infection of a permissive cell by hepacivirus-like particles according to any of claims 14 to 23.

35. A diagnostic kit useful for the method of claim 34, comprising a hepacivirus-like particle according to any of claims 14 to 23 and appropriate means of detection of said immune complexes.

36. Vaccine composition comprising a hepacivirus-like particle according to any of claims 14 to 24 and a pharmaceutically acceptable carrier.

37. A method for in vitro transferring a transgene of interest in a hepatic cell comprising infecting a cell with a hepacivirus-like particle as described in any of claims 14 to 24, wherein the hepacivirus-like particle carries a transgene of interest.

38. Use of a hepacivirus-like particle according to any of claims 14 to 24, that carries a transgene of interest, for the preparation of a medicament for the prevention or treatment of a disease in a patient, wherein the hepacivirus-like particle allows the transfer of the transgene of interest into a cell of the patient, and encodes a product that has a prophylactic or therapeutic effect against the disease.

39. A transformed host cell that contains:
a first nucleic acid sequence comprising a packaging competent retrovirus-derived genome;
a second nucleic acid sequence comprising a cDNA encoding the core proteins from said retrovirus; and
a third nucleic acid sequence comprising a cDNA encoding a polyprotein comprising successively a signal peptide from a type 1 membrane protein, and a hepacivirus E1 protein and/or a hepacivirus E2 protein.

40. The transformed host cell according to claim 39 wherein said third nucleic acid sequence comprising a cDNA encoding a polyprotein comprising successively a hepacivirus core protein, and a hepacivirus E1 protein and/or a hepacivirus E2 protein.

41. The transformed host cell according to claim 39 or 40, wherein said third nucleic acid sequence comprises a cDNA encoding a polyprotein that further comprises a HCV p7 protein.

42. The transformed host cell according to any of claims 39 to 41, wherein said hepacivirus is HCV.

43. A method of ex vivo screening of molecules capable of interfering with hepacivirus entry in cells comprising comparing the level of fusion of a transformed host cell according to any of claims 39 to 42 to a target host cell, in the presence or the absence of a candidate molecule.

44. The method according to claim 43, comprising the steps consisting of:
co-culturing said transformed host cell with a target host cell, in the absence or presence of a candidate molecule, under conditions that allow syncytia formation, i.e. cell-cell fusion, and hepacivirus-like particle entry in target host cell in the absence of any candidate molecule;
assessing syncytia formation in the absence or in the presence of said candidate molecule;
comparing syncytia formation measured in presence of said candidate molecule with syncytia formation measured in absence of any candidate molecule;
identifying as a molecule capable of interfering with hepacivirus entry the candidate molecule for which syncytia formation, as measured in the presence of said molecule, is decreased as compared to syncytia formation measured in the absence of any candidate molecule.

45. The method, according to any of claims 29 to 34, 37, and 42 to 44, wherein said hepacivirus is HCV.
